(19) United States
(12) Patent Application Publication
(10) Pub. No.: US 2025/0325556 A1
(43) Pub. Date: Oct. 23, 2025

(54) COMPOSITIONS FOR LOCAL BONE FORMATION

(71) Applicant: OssiFi Therapeutics LLC, Overland Park, KS (US)

(72) Inventors: Debra Ellies, Parkville, MO (US); F. Scott Kimball, Lake Quivira, KS (US)

(21) Appl. No.: 18/965,641

(22) Filed: Dec. 2, 2024

Related U.S. Application Data
(63) Continuation of application No. 18/058,450, filed on Nov. 23, 2022, now abandoned.
(60) Provisional application No. 63/282,629, filed on Nov. 23, 2021.

(51) Int. Cl. A61K 31/5377; A61K 9/00; A61K 45/06

(52) U.S. Cl. CPC A61K 31/5377; A61K 9/0024; A61K 45/06

(57) ABSTRACT

Provided herein are compositions for bone formation, comprising a scaffold of hydroxyapatite (HA) and tricalcium phosphate (TCP) in a ratio of from 0/100 to 15/85, collagen, and bioactive glass, wherein the bioactive glass is uniformly dispersed in both interior and surface portions of the scaffold, and wherein the composition is sterilized and packaged.

### CROSS-REFERENCES TO RELATED APPLICATIONS

This application is a continuation of the U.S. Non-provisional application Ser. No. 18/058,450 filed on Nov. 23, 2022 which claims the benefit under 35 U.S.C. § 119(e) of U.S. Provisional Application No. 63/282,629 filed on Nov. 23, 2021, each of which is incorporated by reference herein in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Bone homeostasis involves the counterbalancing processes of bone formation and bone resorption. Increased bone resorption and loss of bone homeostasis is associated with a number of diseases and disorders, such as osteoporosis and Paget's disease. All FDA approved therapeutics for treating low bone density, except Teriparatide, do so by stopping bone resorption, hence antiresoptives. Antiresorptives act on the osteoclast cell by stopping them from resorbing the bone.

It is well known in the art that bone can be formed by two processes; one of which is mediated though a chondrocyte cartilage intermediate, (endochondral) and the other is a direct process that stimulates the osteoblast cells (intramembranous). The endochondral process involves chondrocytes/cartilage cells which die and leave a void space which become occupied by osteoblast cells that calcified on the surface of the chondrocyte cartilage calcification. During the resorption process the osteoclasts resorb this cartilage calcification leaving a clean non-cartilage bone mineral behind. The endochondral process is present during the rudimentary formation and growth of long bones, and during the cartilage callus process of bone fractures. Endochondral process begins when mesenchymal stem cells differentiate into chondrocytes creating cartilage. Whereas the intramembranous process occurs during new bone growth stage of bone fractures and formation of bones of the head. Intramembranous process occurs when mesenchymal stem cells differentiate into an osteoblast cell. Unlike cartilage, which is an elastic tissue, bone is hard and rigid. Two very different cellular processes (osteoblasts vs chondrocytes) involving different molecular (WNT vs BMP) and cellular mechanisms (osteoblasts vs chondrocytes).

It is well understood that osteoblast cells are responsible for secreting the bone mineral that causes increases in bone density. To date, only teriparatide was known to stimulate the osteoblast cell to increase mineral deposit, albeit indirectly through the Wnt pathway.

It is desirable to cause osteoblast mineral deposition (bone formation) for treatment of a wide variety of disparate disorders in mammals including simple aging, bone degeneration and osteoporosis, fracture healing, osteogenesis imperfecta, HPP, fusion of two bones or arthrodesis across a joint, degenerative joint disease, degenerative gum disease or periodontitis, any low bone density disorder, etc., as well as for successful installation of various medical orthopedic and periodontal implants such as screws, rods, titanium cage or other cage for spinal fusion, hip joints, knee joint, ankle joints, shoulder joints, dental implants, bone grafts, plates and rods, etc.

A current unmet medical need using current approved therapies in the field of non-union fractures is the desire to improve the poor healing observed in long bone large defects consisting of a large void between bone fracture ends. The use of demineralized bone or similar osteoconductive material, which is known in the art, has not, in many cases, resulted in the desired effects of fusing long bones across small or larger voids.

A variety of materials are available to assist bone formation, including a sterilized tricalcium phosphate (TCP) scaffold with a type 1 collagen (Col1) binder. However, this material provides incomplete or inconsistent mineral levels in vitro. Another system involves a sterilized TCP scaffold with a Col1 binder that also encases bioactive glass into the scaffold. Again, this materials fails to provide full or consistent mineral levels in vitro. Yet another material available is a two part sterilized system. Component 1 is TCP scaffold with Col1 binder. Component 2 is 45S5 bioactive glass (90-150 micron particulate size). Components 1 and 2 must be combined by the surgeon in the surgical suite and can lead to unequal distribution of bioactive glass in the scaffold system and variability.

Thus, there remains a need in the art for new compositions and methods of treating bone disorders, bone fractures and related issues. The present invention meets these and other needs.

### BRIEF SUMMARY OF THE INVENTION

In view of the shortcomings of available materials for bone formation, provided herein in one aspect, is a sterilized composition for bone formation, comprising:
a) a scaffold comprising hydroxyapatite and tricalcium phosphate in a ratio of from 0/100 to 15/85, collagen, and bioactive glass, wherein the bioactive glass is uniformly dispersed in both interior and surface portions of the scaffold; and
b) a compound of Formula I:

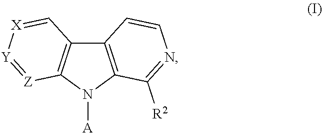

or a salt, hydrate, prodrug, or isomer thereof, wherein the variables X, Y, Z, A and R² have the meanings described below; and wherein the composition is sterilized and packaged.

Surprisingly, such compositions provide enhanced mineralization and bone growth at bone-forming sites.

In another aspect, provided herein are compositions for bone formation, comprising a scaffold of hydroxyapatite (HA) and tricalcium phosphate (TCP) in a ratio of from 0/100 to 15/85, collagen, and bioactive glass, wherein the bioactive glass is uniformly dispersed in both interior and surface portions of the scaffold, and wherein the composition is sterilized and packaged.

In yet another aspect, provided herein are methods of promoting bone formation a subject in need thereof, comprising locally administering to the subject an effective amount of one or more of the compositions described herein.

### BRIEF DESCRIPTION OF THE DRAWINGS

FIG. 1 shows ARS mineralization is reduced in presence of 15% HA/85% TCP Scaffold. The first column represents wildtype mineralization, whereby the cells have no additives (sclerostin, O20221-1: 4-(2-(2-methoxy-8-(trifluoromethyl)-9H-pyrrolo[2,3-b:5,4-c′]dipyridin-9-yl)ethyl)morpholine, scaffold). Addition of ectopic sclerostin inhibits normal mineralization (column 3). The positive control O20221-1 in presence of sclerostin restores normal mineral (compare column 3 and 5). 15% HA/85% TCP scaffold alone is unable produce 100% wildtype mineral (compare column 1 with column 2) and is unable to restore 100% wildtype mineral in presence of sclerostin (column 4). O20221-1 loaded 15% HA/85% TCP scaffold is unable to restore normal mineral (column 6 compared to column 1).

FIG. 2 shows ARS mineralization is reduced by increasing 15% HA/85% TCP scaffold size. The first column represents wildtype mineralization, whereby the cells have no additives (sclerostin, O20221-1, scaffold or granules). In column 2, Sclerostin inhibits wildtype mineralization. The addition of O20221-1 in presence of sclerostin restores normal mineral (Column 3). Decreasing the size of the scaffold from 0.12 cc-0.01 cc restores the reduced mineral (column 7) back to wildtype mineral levels (dose response & columns 4-7). 15% HA/85% TCP scaffold shows decrease in mineral as scaffold size increases from 0.01 cc to 0.12 cc (compare column 4 to 1).

FIG. 3 shows ARS mineralization is reduced by increasing percentage of HA content. The first column represents wildtype mineralization, whereby the cells have no additives (sclerostin, O20221-1, scaffold or granules). In column 2, Sclerostin inhibits wildtype mineralization. The addition of O20221-1 in presence of sclerostin restores normal mineral (Column 3). Addition of 15% HA/85% TCP scaffold alone shows a decrease in wild type mineral (compare column 4 to 1). Increasing concentration (percentages from 5-50%) of HA decreases wild type mineral produced (dose response & columns 5-8).

FIG. 4 is a table summarizing surprising results. The first 5 columns show the effect of variable HA content from 0-50% on mineralization 100% to 0%. The low mineralization of the scaffold in presence of sclerostin and O20221-1 can be restored by the addition of non-encased bioactive glass (column 6 & 7 compared to column 2 & 3).

### DETAILED DESCRIPTION OF THE INVENTION

### I. Introduction

Bone mass homeostasis and bone remodeling involve the counterbalancing processes of bone formation (osteoblast cell depositing mineral, an anabolic process) and bone resorption (osteoclast cell resorbing mineral, bone loss, a catabolic process). These two processes are coupled in a healthy bone. In bone formation, osteoblasts synthesize bone matrix and regulate mineralization, and then terminally differentiate into osteocytes or bone lining cells. In bone resorption, a different cell type—osteoclasts—remove mineralized bone matrix and break up the organic bone to release calcium in the serum. See, e.g., Kular et al., Clinical Biochemistry 45:863-873 (2012).

The osteoblasts (bone formation cells) and osteoclasts (bone resorption cells) are regulated by different mechanisms. Osteoclast cell differentiation is regulated or controlled by the osteoblast (Glass et al., Dev Cell 8:751-764 (2005)) or other hormones like PTH, calcitonin, or IL6. In contrast, osteoblast cell differentiation or activity is not regulated or controlled by osteoclast cells, but rather are controlled by different signals, like CPFA, hedgehog, WNT/LRP, and sclerostin. Bone formation can occur via endochondral ossificiation or intramembranous ossification (sclerostin). In intramembranous ossification, bone forms directly through the stimulation of osteoblast/osteocyte bone cells. In endochondral ossification, bone formation occurs by way of a cartilage template, which increases the amount of time that it takes bone to form. BMP signaling is implicated in endochondral ossification, whereas Wnt signaling has been shown to be involved in both endochondral and intramembranous ossification.

Under normal healthy conditions, bone remodeling (or bone homeostasis) involves the degradation of old bone (via osteoclast cells) and the repair or replacement of the old bone with new bone (via osteoblast cells). When this homeostasis is disrupted and bone resorption exceeds bone formation, i.e. diseased bone state, the results uncouple bone resorption from bone formation. Increased bone resorption leads to decreased bone mass or density (loss of trabecular bone) and greater bone fragility (less bone strength). A number of diseases and conditions are associated with increased bone resorption or poor bone strength/quality, including osteoporosis, osteogenesis imperfecta, Paget's disease of bone, metabolic bone disease, bone changes secondary to cancer, and other diseases characterized or associated with low bone density.

Diseases caused by increased bone resorption are associated with decreased bone mass density and greater bone fragility and are frequently treated with antiresorptive agents such as bisphosphonates, denosumab, prolia, alendronate, cathepsin K modulators, RankL inhibitors, estrogens, cathepsin K inhibitors, selective estrogen receptor modulators, and Vitamin D, to name but a few. These agents function by preventing or inhibiting osteoclast cell bone resorption, either directly or indirectly. However, these agents do not promote the formation of new bone by the osteoblast cell (i.e., anabolic bone formation); in contrast, administration of one dose of an anabolic agent normally results in annual cumulative increase of >8% from baseline in bone formation in lumbar vertebra of humans (Padhi et al. 2010 JBMR). Administration of an antiresorptive does result in a modest increase in bone density the first year of <7% but thereafter the increase in bone density is <3.5% with an annual cumulative increase of <10%. Therefore, although a fragile osteoporotic bone that is treated with an antiresorptive agent will result in the fragile bone not getting more fragile, the fragile bone will not be stronger or have increased strength because the antiresorptive agent does not promote new bone growth by depositing more bone mineral to increase bone density. In contrast, an agent that promotes anabolic bone growth, for example, by stimulating the activity of osteoblasts, promotes the deposition of more bone matrix, or if proliferation were stimulated, the agent would result in more osteoblast cells, thus resulting in more bone cells to bridge a gap to fuse two bones. Thus, a fragile osteoporotic bone treated with an anabolic bone formation agent will allow the bone not to get more fragile, and also will allow the bone to have more strength due to increased bone formation.

With out being bound to a particular theory, if one thinks of the bone as a bathtub, the drain is reminiscent of bone loss or resorption and the faucet reminiscent of the bone being added or bone formation. Both the faucet and drain are adding and removing at the same rate (coupled) until one ages or a disease strikes causing either the faucet to be turned down or the drain to be increased in size. Perturbations such as these result in an imbalance (uncoupling) of formation/resorption causing bone density to become lowered. For example, imagine a sponge that has an outer core and on the inside is made of fibers stretching from one end to the other. During bone resorption these fibers are removed, and if bone resorption is occurring at a rate faster than bone building or formation then these fibers would be few and the bone would become fragile. It would not take much strength to break a sponge with few inside fibers versus one with many inside fibers. Because the process of bone resorption is well understood, many of the marketed therapeutics stop bone resorption by acting on the osteoclast cells. These include antiresorptive agents such as Cathepsin K inhibitors, Rank Ligand inhibitor, Denosumab, Prolia, Fosamax, Evista, Premarin, osteoprotegerin (OPG) inhibitors, alendronate, selective estrogen receptor modulators (SERMs), bisphosphonates, and other agents acting to stop the activity of the osteoclast cell.

While still considering the analogy of the sponge, to increase bone strength, the number of fibers on the inside of the bone increase in number, thickness and strength to increase bone strength overall. However, it is not possible to increase bone strength by acting on the bone resorbing cell, the osteoclast. Thus, one needs to focus on the bone forming osteoblast cell. Unlike bone resorption, bone formation is not well understood and, until recently, only one systemic therapeutic (teriparatide) and one surgical implant (Infuse with BMP protein) has been marketed to promote bone formation. However, BMP product acts to increase chondrocytes and promote cartilage production first before undergoing endochondral bone formation. This process sometimes leads to the chondrocytes then being replaced by osteoblasts.

Intermittent teriparatide administration increases bone density systemically by activation of PKA which then phosphorylates LRP and activates the WNT pathway (Wan et al., Genes Dev. 22(21): 2968-2979 (2008)). This increase in bone density occurs along already laid down trabeculae within the bone matrix. The osteoblast cells lining the trabeculae secrete mineral onto the existing trabecular bone thus increasing the amount of mineral and density of the trabeculae.

When a bone void exists whereby a large segment of bone is removed or missing causing non-union of the bone or a critical size defect. The bone is unable to heal itself across a large gap. The addition of BMP to the site causes the pluripotent cells to differentiate into chondrocytes/cartilage and produce a cartilage callus. The ability of the gap to be filled by bone instead of cartilage would require osteoblast bone cells to undergo proliferation to fill the gap and then to deposit mineral to fill the void.

Bioactive glasses have been considered as scaffold materials for bone repair and have an ability to foster the growth of bone cells, and to bond strongly with both hard and soft tissues. Upon implantation, bioactive glasses undergo specific reactions, leading to the formation of an amorphous calcium phosphate (ACP) or crystalline hydroxyapatite (HA) phase on the surface of the glass, which is responsible for their strong bonding with the surrounding tissue. Bioactive glasses are also reported to release ions that activate expression of osteogenic genes, and to stimulate angiogenesis. See, Fu, Q., Mater Sci Eng C Mater Biol Appl. 2011 Oct. 10; 31(7): 1245-1256.

Without being bound to a particular theory, it is believed that compositions described herein operate as SOST (Sclerostin) and/or WISE antagonists that function by modulating the Wnt/LRP and/or BMP signaling pathways. SOST and WISE are proteins that are believed to modulate bone formation by either binding to the Wnt co-receptor LRP, thereby inhibiting the Wnt signaling pathway, or by binding to BMP and inhibiting BMP activity, via different amino acid sequences or domains within Sclerostin. By neutralizing the inhibitory effects of SOST and/or WISE proteins on the Wnt pathway, the compounds and compositions of the present invention restore Wnt signaling and promote bone formation/growth. Thus, in one aspect, the present invention provides compositions, and methods for promoting bone formation in a subject. The bone formation is generally considered as local bone formation. The compositions of the present invention can be administered locally and optionally can be administered sequentially or in combination with one or more other therapeutic agents. In another aspect, the present invention provides implantable devices such as orthopedic hardware or as structural scaffolds for allowing osteoblast/osteocytes to migrate into the scaffold and deposit bone mineral and also for delivering bone formation agents, e.g., for promoting bone formation at the site of implantation.

### II. Definitions

As used herein, the term “pharmaceutically acceptable excipient” refers to a substance that aids the administration of an active agent to and absorption by a subject. Pharmaceutically acceptable excipients useful in the present invention include, but are not limited to, binders, fillers, disintegrants, lubricants, coatings, sweeteners, flavors and colors. One of skill in the art will recognize that other pharmaceutical excipients are useful in the present invention.

As used herein, the term “alkyl” refers to a straight or branched, saturated, aliphatic radical having the number of carbon atoms indicated. For example, C₁-C₆ alkyl (or C₁₋₆ alkyl) includes, but is not limited to, methyl, ethyl, propyl, butyl, pentyl, hexyl, isopropyl, isobutyl, sec-butyl, tert-butyl, etc.

Alkylene represents either straight chain or branched alkylene of 1 to 7 carbon atoms, i.e. a divalent hydrocarbon radical of 1 to 7 carbon atoms; for instance, straight chain alkylene being the bivalent radical of Formula —(CH₂)ₙ—, where n is 1, 2, 3, 4, 5, 6 or 7. Preferably alkylene represents straight chain alkylene of 1 to 4 carbon atoms, e.g. a methylene, ethylene, propylene or butylene chain, or the methylene, ethylene, propylene or butylene chain mono-substituted by C₁-C₃ alkyl (preferably methyl) or disubstituted on the same or different carbon atoms by C₁-C₃ alkyl (preferably methyl), the total number of carbon atoms being up to and including 7. One of skill in the art will appreciate that a single carbon of the alkylene can be divalent, such as in —CH((CH₂)ₙCH₃)—, wherein n=0-5.

As used herein, the term “alkoxy” or “—O-alkyl” refers to alkyl with the inclusion of an oxygen atom, for example, methoxy, ethoxy, etc. “Haloalkoxy” is as defined for alkoxy where some or all of the hydrogen atoms are substituted with halogen atoms. For example, halo-substituted-alkoxy includes trifluoromethoxy, etc.

The term “hydroxyalkyl” or “alkyl-OH” refers to an alkyl group, as defined above, where at least one of the hydrogen atoms is replaced with a hydroxy group. As for the alkyl group, hydroxyalkyl groups can have any suitable number of carbon atoms, such as C₁₋₆. Exemplary hydroxyalkyl groups include, but are not limited to, hydroxymethyl, hydroxyethyl (where the hydroxy is in the 1- or 2-position), hydroxypropyl (where the hydroxy is in the 1-, 2- or 3-position), etc.

As used herein, the term “alkenyl” refers to either a straight chain or branched hydrocarbon of 2 to 6 carbon atoms, having at least one double bond. Examples of alkenyl groups include, but are not limited to, vinyl, propenyl, isopropenyl, butenyl, isobutenyl, butadienyl, pentenyl or hexadienyl.

As used herein, the term “alkynyl” refers to either a straight chain or branched hydrocarbon of 2 to 6 carbon atoms, having at least one triple bond. Examples of alkynyl groups include, but are not limited to, acetylenyl, propynyl or butynyl.

As used herein, the term “halogen” refers to fluorine, chlorine, bromine and iodine.

As used herein, the term “haloalkyl” refers to alkyl as defined above where some or all of the hydrogen atoms are substituted with halogen atoms. Halogen (halo) preferably represents chloro or fluoro, but may also be bromo or iodo. For example, haloalkyl includes trifluoromethyl, fluoromethyl, etc. The term “perfluoro” defines a compound or radical which has at least two available hydrogens substituted with fluorine. For example, perfluoromethane refers to 1,1,1-trifluoromethyl, and perfluoromethoxy refers to 1,1,1-trifluoromethoxy.

As used herein, the term “heteroalkyl” refers to an alkyl group having from 1 to 3 heteroatoms such as N, O and S. Heteroalkyl groups have the indicated number of carbon atoms where at least one non-terminal carbon is replaced with a heteroatom. Additional heteroatoms can also be useful, including, but not limited to, B, Al, Si and P. The heteroatoms can also be oxidized, such as, but not limited to, —S(O)— and —S(O)₂—. For example, heteroalkyl can include ethers, thioethers and alkyl-amines. Heteroalkyl groups do not include peroxides (—O—O—) or other consecutively linked heteroatoms.

As used herein, the term “oxo” refers to a double bonded oxygen (=O).

As used herein, the term “cycloalkyl” refers to a saturated or partially unsaturated, monocyclic, fused bicyclic or bridged polycyclic ring assembly containing from 3 to 12 ring atoms, 3 to 8, 3 to 6, or the number of atoms indicated. For example, C₃₋₈ cycloalkyl includes cyclopropyl, cyclobutyl, cyclopentyl, cyclohexyl, and up to cyclooctyl. The cycloalkyl groups of the present invention are optionally substituted as defined below.

As used herein, the terms “heterocycle,” “heterocycloalkyl,” and “heterocyclyl” refer to a ring system having from 3 ring members to about 20 ring members and from 1 to about 5 heteroatoms such as N, O and S. Additional heteroatoms can also be useful, including, but not limited to, B, Al, Si and P. The heteroatoms can also be oxidized, such as, but not limited to, —S(O)— and —S(O)₂—. The term heterocycle includes monocyclic, fused bicyclic, and bridged cyclic moieties. For example, heterocycle includes, but is not limited to, tetrahydrofuranyl, tetrahydrothiophenyl, morpholino, pyrrolidinyl, pyrrolinyl, imidazolidinyl, imidazolinyl, pyrazolidinyl, pyrazolinyl, piperazinyl, piperidinyl, indolinyl, quinuclidinyl hexahydro-1H-furo[3,4-c]pyrrolyl and 1,4-dioxa-8-azaspiro[4.5]dec-8-yl. The heterocycloalkyl groups of the present invention are optionally substituted as defined below.

Substituents for the cycloalkyl and heterocyclyl groups are varied and are independently selected from: -halogen, C₁₋₈alkyl, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —S(O)R′, —S(O)₂R′, —S(O)₂NR′R″, perfluoro(C₁-C₄)alkoxy, and perfluoro(C₁-C₄)alkyl, in a number ranging from zero to the total number of open valences on the ring system; and where R′, R″ and R′″ are independently selected from hydrogen, (C₁-C₈)alkyl and C₃₋₈ heteroalkyl, and phenyl.

As used herein, a group “linked via a carbon atom” refers to a linkage between a carbon atom of the referenced group and the rest of the molecule. A group “linked via a nitrogen atom” refers to a linkage between a nitrogen atom of the referenced group and the rest of the molecule.

By way of example only, a heterocyclyl group linked via a carbon atom may be:

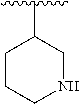 [SMILES: *C1CCCNC1]

where the wavy line indicates the point of attachment to the rest of the molecule. By way of example only, a heterocyclyl group linked via a nitrogen atom may be:

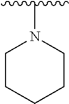 [SMILES: *N1CCCCC1]

where the wavy line indicates the point of attachment to the rest of the molecule.

As used herein, where a referenced compound is an N-oxide, it comprises an N—O bond with three additional bonds to the nitrogen, i.e., an N-oxide refers to a group R₃N⁺—O⁻. By way of example only, N-oxides may include:

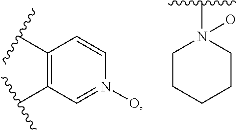

and the like.

As used herein, the term “aryl” refers to a monocyclic or fused bicyclic, tricyclic or greater, aromatic ring assembly containing 6 to 16 ring carbon atoms. For example, aryl may be phenyl, benzyl or naphthyl, preferably phenyl. “Arylene” means a divalent radical derived from an aryl group. Aryl groups can be mono-, di- or tri-substituted by one, two or three radicals as described below.

Substituents for the aryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′C(O)NR″R′″, —NHC(NH₂)=NH, —NR′C(NH₂)=NH, —NHC(NH₂)═NR′, —S(O)R′, —S(O)₂R′, —S(O)₂NR′R″, alkylenedioxy, heteroaryl, —C₁₋₂alkylene-heteroaryl, heterocyclyl, C₁₋₂alkylene-heterocyclyl, phenyl, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and C₃₋₈ heteroalkyl, and phenyl. Alkylenedioxy is a divalent substitute attached to two adjacent carbon atoms of phenyl, e.g. methylenedioxy or ethylenedioxy. Oxy-C₂-C₃-alkylene is also a divalent substituent attached to two adjacent carbon atoms of phenyl, e.g. oxyethylene or oxypropylene.

Examples of substituted phenyl groups include, but are not limited to 4-chlorophen-1-yl, 3,4-dichlorophen-1-yl, 4-methoxyphen-1-yl, 4-methylphen-1-yl, 4-aminomethylphen-1-yl, 4-methoxyethylaminomethylphen-1-yl, 4-hydroxyethylaminomethylphen-1-yl, 4-hydroxyethyl(methyl)aminomethylphen-1-yl, 3-aminomethylphen-1-yl, 4-Nacetylaminomethylphen-1-yl, 4-aminophen-1-yl, 3-aminophen-1-yl, 2-aminophen-1-yl, 4-phenylphen-1-yl, 4-(imidazol-1-yl)phenyl, 4-(imidazol-1-ylmethyl)phen-1-yl, 4-(morpholin-1-yl)phen1yl, 4-(morpholin-1-ylmethyl)phen-1-yl, 4-(2-methoxyethylaminomethyl)phen-1-yl and 4-(pyrrolidin-1-ylmethyl)phen-1-yl, 4-(thiophenyl)phen-1-yl, 4-(3-thiophenyl)phen-1-yl, 4-(4-methylpiperazin-1-yl)phen-1-yl, and 4-(piperidinyl)phenyl and 4-(pyridinyl)phenyl optionally substituted in the heterocyclic or heteroaryl ring.

As used herein, the term “heteroaryl” refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl. Heteroaryl moieties can be optionally substituted, as defined below.

Preferably, heteroaryl is pyridyl, indolyl, quinolinyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazolyl, indazolyl, or any of the radicals substituted, especially mono- or di-substituted.

Substituents for the heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′C(O)NR″R′″, —NHC(NH₂)=NH, —NR′C(NH₂)=NH, —NHC(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and C₃₋₈ heteroalkyl, and phenyl.

As used herein, the terms “ring members” and “ring vertices” are intended to have the same meaning. For example, a six membered ring has six ring vertices.

As used herein, the term “salt” refers to acid or base salts of the compounds used in the methods of the present invention. Illustrative examples of pharmaceutically acceptable salts are mineral acid (hydrochloric acid, hydrobromic acid, sulfuric acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris- (hydroxymethyl)-methyl-ammonium salts.

Similarly, acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

As used herein, the term “bioactive glass” or “bioglass” refers to a silica composite useful in bone grafting, repair of periodontal defects, and cranial and maxillofacial repair. A common bioactive glass is Bioglass 45S5 or calcium sodium phosphosilicate, is a bioactive glass specifically composed of 45 wt % SiO2, 24.5 wt % CaO, 24.5 wt % Na2O, and 6.0 wt % P₂O₅, which is commercially available as NovaMin. Other bioactive glass compositions include S53P4: 53 wt % SiO2, 23 wt % Na2O, 20 wt % CaO and 4 wt % P2O5; 58S: 58 wt % SiO2, 33 wt % CaO and 9 wt % P2O5; 70S30C: 70 wt % SiO2, 30 wt % CaO; 13-93: 53 wt % SiO2, 6 wt % Na2O, 12 wt % K2O, 5 wt % MgO, 20 wt % CaO, 4 wt % P2O5.

As used herein, the term “calcium salt” refers to salts containing calcium. Examples of calcium salts include, but are not limited to, calcium acetate, calcium aluminates, calcium aluminosilicate, calcium arsenate, calcium borate, calcium bromide, calcium carbide, calcium carbonate, calcium chlorate, calcium chloride, calcium citrate, calcium citrate malate, calcium cyanamide, calcium dihydrogen phosphate, calcium fluoride, calcium formate, calcium glubionate, calcium glucoheptonate, calcium gluconate, calcium glycerylphosphate, calcium hexaboride, calcium hydride, calcium hydroxide, calcium hypochlorite, calcium inosinate, calcium iodate, calcium iodide, calcium lactate, calcium lactate gluconate, calcium magnesium acetate, calcium malate, calcium nitrate, calcium nitride, calcium oxalate, calcium oxide, calcium pangamate, calcium peroxide, calcium phosphate, calcium phosphide, calcium propionate, calcium pyrophosphate, calcium silicate, calcium silicide, calcium sorbate, calcium stearate, calcium sulfate, calcium sulfide, calcium tartrate, calcium(I) chloride, dicalcium citrate, dicalcium phosphate, dodecacalcium hepta-aluminate, tricalcium aluminate, tricalcium phosphate and triple superphosphate. One of skill in the art will appreciate that other calcium salts are useful in the present invention.

As used herein, the term “hydrate” refers to a compound that is complexed to at least one water molecule. The compounds of the present invention can be complexed with from 1 to 10 water molecules. The term “hydrate” also includes hemi-hydrates, where there are two compounds for every water molecules in the complex.

Certain compounds used in the present invention possess asymmetric carbon atoms (optical centers) or double bonds; the racemates, diastereomers, geometric isomers and individual isomers are all intended to be encompassed within the scope of the present invention.

As used herein, the term “subject” refers to animals such as mammals, including, but not limited to, primates (e.g., humans), cows, sheep, goats, horses, dogs, cats, rabbits, rats, mice and the like. In certain embodiments, the subject is a human.

As used herein, the terms “therapeutically effective amount” or “therapeutically sufficient amount” or “effective or sufficient amount” refer to an amount that produces therapeutic effects for which it is administered. The exact amount will depend on the purpose of the treatment, and will be ascertainable by one skilled in the art using known techniques.

As used herein, the term “site of injury or localized condition” refers to a specific location in the subject's body that is in need of treatment by the method of the present invention. For example, the injury can be a fracture and the localized condition can be a disease state (such as osteoporosis, etc.) that is limited to a particular location in the subject's body, such as a particular bone, joint, digit, hand, foot, limb, spine, head, torso, etc. In some embodiments, the site of injury or localized condition is a surgical implantation site.

As used herein, the term “promoting bone formation” refers to stimulating new bone formation, growing bone across a joint or gap, enhancing or hastening bone formation, and/or increasing bone density or bone mineral content. In some embodiments, a compound promotes bone formation if it increases the amount of bone in a sample by at least 3%, 4%, 5%, 6%, 7%, 8%, 9%, 10%, 11%, 12%, 13%, 14%, 15%, 16%, 17%, 18%, 19%, 20%, or more relative to a control sample (e.g., a sample that has not been contacted with the compound).

As used herein, the term “arthrodesis” refers to the artificial induction of joint ossification between two bones and/or across a joint, often via surgery. Arthrodesis can be accomplished via bone graft, metal implants or the use of synthetic bone substitutes, among others.

As used herein, the term “bone autograft” refers to the grafting of a subject's own bone.

As used herein, the term “bone allograft” refers to the grafting of bone from one person to another person.

As used herein, the term “antiresorptive drug” refers to drugs that slow or block the resorption of bone and/or that act on the osteoclast cell.

As used herein, the term “bone related disease characterized by low bone mass” refers to bone having a T-score less than −0.5. Other methods of determining low bone mass are known by one of skill in the art.

As used herein, the term “bone fracture” refers to bone that has been cracked, fractured, or broken in one or several locations along the bone. In some embodiments, the term “bone fracture” also includes a segment of the bond missing.

As used herein, the term “spinal fusion” refers to a surgical technique for combining or fusing two or more vertebrae.

As used herein, the term “scaffold” refers to a segment of a device that can be implanted in a subject (implantable portion). The scaffold can be prepared from a variety of different materials, as described herein, and further including metals, ceramics, polymers and inorganic materials, such as described below. Non-limiting examples of scaffolds include blocks, cubes, spheres, putty, liquid cement, strips, as well as granules. The scaffold can be coated with a variety of materials that promote bone growth. In some embodiments, the entire device comprises an implantable scaffold. For example, in some embodiments, an entire device as described herein can be implanted at a surgical site and the surgical site can be closed over the device.

As used herein, the term “external coating” refers to a coating of the structural support that can cover only a portion of the structural support (partial external coating) or cover the entire structural support. For example, the partial external coating can completely cover only the implantable portion of the structural support.

As used herein, the term “weakened bone,” “low bone density,” or “low bone mass” refers to bone that has a T score of less than −0.5 (less than 0.9 g/cm2).

As used herein, the term “demineralized bone” refers to bone from which the inorganic mineral have been removed. The remaining organic collagen material may contain the osteoinductive growth factors. These growth factors include bone morphogenetic proteins that induce cartilage which then ossify via endochondral ossification to generate new bone formation. Demineralized bone often comes in the form of “demineralized bone matrix (DBM).” DBM can be made by fresh frozen or freeze dried bulk bone allograft, or can be made from mild acid extraction of cadaveric bone that removes the mineral phase, leaving collagen, growth factors, and noncollagenous proteins that offer the intrinsic properties of osteoconduction. DBM can also be processed in a variety of ways, ultimately resulting in a powder that is mixed with a carrier to provide the optimum handling characteristics desired by a surgeon. DBM is clinically available in gels, pastes, putty, and fabrics that have been tailored to meet the needs of the surgical procedure. Some DBM are mixed with antibiotics prior to the surgical procedure.

As used herein, the term “diabetes” refers to a condition primarily characterized by a body's inability to produce sufficient amounts of insulin, a hormone produced in the pancreas. When released in the blood steam, insulin induces cellular glucose uptake. As such, insufficient amounts of insulin result in elevated blood glucose levels in affected individuals. A person of skill in the art will recognize that the body's inability to produce sufficient amounts of insulin can be a characteristic of both Type 1 and Type 2 Diabetes.

As used herein, the term “osteoconductive matrix” refers to a material that can act as an osteoconductive substrate (i.e., permits bone growth) and has a scaffolding structure on which infiltrating cells can attach, proliferate, and participate in the process of producing osteoid, the organic phase of bone, culminating in osteoneogenesis, or new bone formation. The terms “matrix” and “scaffold” interchangeably refer to a structural component or substrate intrinsically having a 3 dimensional form upon which the specific cellular events involved in bone formation will occur. The osteoconductive matrix allows for the ingrowth of host capillaries, perivascular tissue and osteoprogenitor cells. In some embodiments, an osteoconductive matrix includes an “osteoinductive agent” for providing osteogenic potential. An osteoinductive agent, as used herein, is an agent that stimulates the host to multiply bone cells, thus producing more bone osteoid.

As used herein, the terms “treat,” “treating,” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom or condition. The treatment or amelioration of symptoms can be based on any objective or subjective parameter; including, e.g., the result of a physical examination.

As used herein, the term “RankL inhibitor” refers to compounds or agents that inhibit the activity of RankL. RankL (Receptor Activator for Nuclear Factor×B Ligand), is important in bone metabolism by activating osteoclasts. RankL inhibitors include, but are not limited to, the human monoclonal antibody denosumab. One of skill in the art will appreciate that other RankL inhibitors are useful in the present invention.

As used herein, the term “parathyroid hormone” or “PTH” refers to compounds or agents that act on the PTH receptor to activate the pathway. PTH is important in bone metabolism by activating osteoblasts. PTH include, but are not limited to, Teriparatide, Forteo, and abaloparatide-SC. One of skill in the art will appreciate that other PTH are useful in the present invention.

As used herein, the term “combination therapy” is the use of the present invention in combination either together, or serially before or after the administration of compounds of this invention.

Certain compounds used herein possess asymmetric carbon atoms (optical centers) or double bonds; the racemates, diastereomers, geometric isomers, regioisomers and individual isomers (e.g., separate enantiomers) are all intended to be encompassed within the scope of the present invention. In some embodiments, the compounds of the present invention are a particular enantiomer or diastereomer substantially free of other forms. The compounds of the present invention may also contain unnatural proportions of atomic isotopes at one or more of the atoms that constitute such compounds. For example, the compounds may be radiolabeled with radioactive or non-radioactive isotopes, such as for example deuterium (²H), tritium (³H), iodine-125 (¹²⁵I) or carbon-14 (¹⁴C). All isotopic variations of the compounds of the present invention, whether radioactive or not, are intended to be encompassed within the scope of the present invention.

### III. Compositions

In some embodiments, the present invention provides a sterilized composition for bone formation, comprising:
a) a scaffold comprising hydroxyapatite and tricalcium phosphate in a ratio of from 0/100 to 15/85, collagen, and bioactive glass, wherein the bioactive glass is uniformly dispersed in both interior and surface portions of the scaffold; and
b) a compound of Formula I:

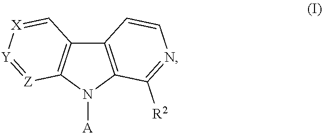

or a salt, hydrate, prodrug, or isomer thereof, wherein
X is selected from CR^{3b} and N, wherein N is optionally oxidized to the corresponding N-oxide;
Y is selected from CR^{3c} and N, wherein N is optionally oxidized to the corresponding N-oxide;
Z is selected from CR^{3d} and N, wherein N is optionally oxidized to the corresponding N-oxide,
provided that at least one of X, Y, and Z is N or the corresponding N-oxide;
A is

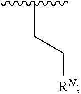

R^{N} is selected from the group consisting of heterocyclyl and heteroaryl, wherein
the heterocyclyl moiety is selected from monocyclic, fused bicyclic, and bridged cyclic, the monocyclic heterocyclyl comprising from 4 to 7 ring members, the fused bicyclic and bridged bicyclic heterocyclyl comprising from 7 to 10 ring members, each heterocyclyl moiety having from 1 to 3 heteroatoms as ring members selected from N, O, and S, wherein each heterocyclyl moiety comprises at least one nitrogen atom as a ring member and is optionally substituted with from 1 to 3 R⁶ moieties,
the heteroaryl moiety comprises from 5 to 10 ring members, wherein at least one ring member is a nitrogen atom and is optionally substituted with from 1 to 3 R⁶ moieties,
each R², R^{3b}, R^{3c} and R^{3d} is independently selected from the group consisting of H, halogen, C₁₋₆ alkyl, C₁₋₆ haloalkyl, C₂₋₆ alkenyl, C₂₋₆ alkynyl, C₁₋₆ alkoxy, C₁₋₆ haloalkoxy, C₁₋₆ alkyl-OH, —O—C₁₋₆ alkyl-OH, C₃₋₆ cycloalkyl-C₁₋₄ alkoxy, and —OH;
R⁶ is selected from the group consisting of —OH, C₁₋₃ alkyl, C₁₋₃ alkyl-OH, —O—C₁₋₃ alkyl, C₃₋₄ heteroalkyl, C₁₋₃ haloalkyl, —O—C₁₋₃ haloalkyl, halogen, and oxo; and wherein the composition is sterilized and packaged.

In some embodiments, the compound used in the sterilized compositions has Formula Ia:

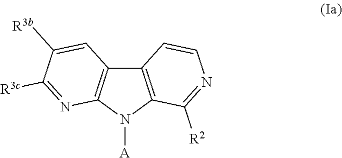

wherein each variable position is as defined in Formula I.

In some embodiments, the compound used in the sterilized compositions has Formula Ib:

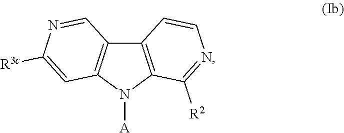

wherein each variable position is as defined in Formula I.

In some embodiments, the compound used in the sterilized compositions has Formula Ic:

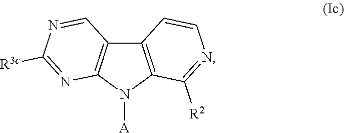

wherein each variable position is as defined in Formula I.

In some embodiments, the compound used in the sterilized compositions has Formula Id:

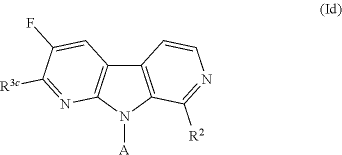

wherein each variable position is as defined in Formula I

In some embodiments, each R² in Formulas I, Ia, Ib, Ic, or Id is independently selected from the group consisting of halogen, C₁₋₆ alkyl, C₁₋₆ haloalkyl, and C₁₋₆ alkoxy. In some embodiments, R² in Formulas I, Ia, Ib, Ic, or Id is C₁₋₆ alkyl or C₁₋₆ haloalkyl. In some embodiments, R² in Formulas I, Ia, Ib, Ic, or Id is CH₃ or CF₃. In some embodiments, R² in Formulas I, Ia, Ib, Ic, or Id is CF₃.

In some embodiments, each R^{3b}, R^{3c} and R^{3d}, when present in Formulas I, Ia, Ib, Ic, or Id is independently selected from the group consisting of H, halogen, C₁₋₆ alkyl, C₁₋₆ haloalkyl, C₁₋₆ alkoxy, C₁₋₆ haloalkoxy. In some embodiments, each R^{3b}, R^{3c} and R^{3d}, when present in Formulas I, Ia, Ib, Ic, or Id is H, halogen, and C₁₋₆ alkoxy. In some embodiments, each R^{3b}, R^{3c} and R^{3d}, when present in Formulas I, Ia, Ib, Ic, or Id is H, F, and methoxy. In some embodiments, at least one of R^{3b}, R^{3c} and R^{3d} when present in Formulas I, Ia, Ib, Ic, or Id is F. In some embodiments, at least one of R^{3b}, R^{3c} and R^{3d} when present in Formulas I, Ia, Ib, Ic, or Id is methoxy. In some embodiments, at least one of R^{3b}, R^{3c} and R^{3d} when present in Formulas I, Ia, Ib, Ic, or Id is F and at least one of R^{3b}, R^{3c} and R^{3d} is methoxy.

In some embodiments, R^{N} of Formulas I, Ia, Ib, Ic, or Id is a heteroaryl. In some embodiments, the heteroaryl moiety comprises from 4 to 8 ring members, wherein at least one ring member is a nitrogen atom and is optionally substituted with from 1 to 3 R⁶ moieties

In some embodiments, R^{N} of Formulas I, Ia, Ib, Ic, or Id is a monocyclic heterocyclyl.

In some embodiments, R^{N} in Formulas I, Ia, Ib, Ic, or Id is

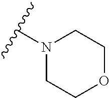 [SMILES: *N1CCOCC1]

In some embodiments, R⁶ in Formulas I, Ia, Ib, Ic, or Id is selected form the group consisting of —OH, C₁₋₃ alkyl, C₁₋₃ alkyl-OH, —O—C₁₋₃ alkyl, C₁₋₃ haloalkyl, —O—C₁₋₃ haloalkyl, halogen, and oxo. In some embodiments, R⁶ in Formulas I, Ia, Ib, Ic, or Id is selected form the group consisting of C₁₋₃ alkyl, —O—C₁₋₃ alkyl, C₁₋₃ haloalkyl, —O—C₁₋₃ haloalkyl, and halogen. In some embodiments, R⁵ is —OH, C₁₋₃ alkyl, or —O—C₁₋₃ alkyl.

In one group of embodiments compounds of Formula I have a structure selected from the following:

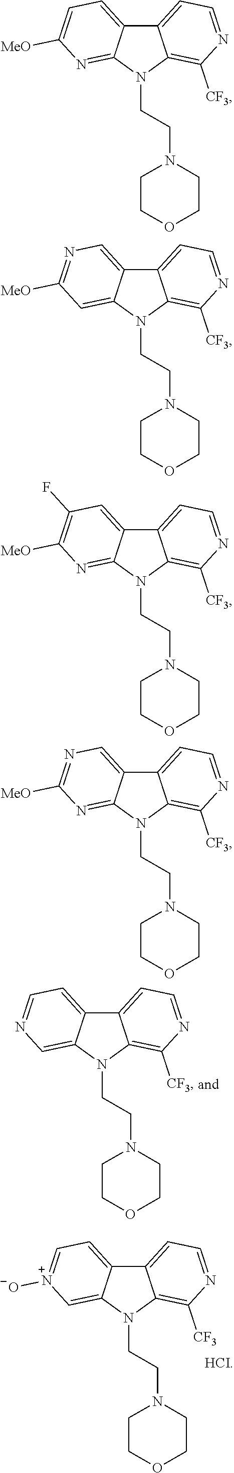

In some embodiments, the compositions described herein comprise a formate salt of a compound according to any of the compounds described above. In some embodiments, the compositions described herein comprise a citrate salt of a compound according to any of the compounds described above. In some embodiments, the compositions described herein comprise a hydrochloride salt of a compound according to any of the compounds described above.

The compositions of the present invention can also include hydrates, solvates, and prodrug forms of the compounds described above (and other bone-forming agents, see for example WO2020/037001, WO2020/037004 and WO2014/153203.

As noted above, the compounds of formula I as used herein can be in the salt form. Salts include, but are not limited, to sulfate, citrate, acetate, oxalate, chloride, bromide, iodide, nitrate, bisulfate, phosphate, acid phosphate, phosphonic acid, isonicotinate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Other salts include, but are not limited to, salts with inorganic bases include alkali metal salts such as sodium salts, and potassium salts; alkaline earth metal salts such as calcium salts, and magnesium salts; aluminum salts; and ammonium salts. Other salts with organic bases include salts with diethylamine, diethanolamine, meglumine, and N,N′-dibenzylethylenediamine. In some embodiments, the present invention provides the hydrochloride salt.

In some embodiments, the compounds of Formula I as used herein comprise nitrogen atoms which are optionally further oxidized, i.e., the compounds are N-oxides. By way of example only, in one instance, a nitrogen atom in a pyrido-indolyl ring system in a compound of Formula I, Ia, Ib, Ic, or Id is oxidized to the corresponding N-oxide.

In some embodiments, the compounds of Formula I as used herein are delivered and/or formulated as prodrugs. In one embodiment, any compound described herein is an ester prodrug. In another embodiment, any compound described herein is an amide prodrug. In further embodiments, the prodrug moieties comprise conjugated groups which allow selective targeting at a bone structure. Examples of such motifs are described in Erez et al., Bioorg. Med. Chem. Lett. 2008, 18, 816-820 and Neale et al., Bioorg. Med. Chem. Lett. 2009, 19, 680-683 and are incorporated herein by reference. Accordingly, contemplated within the scope of embodiments presented herein are estradiol conjugates and/or bisphosphonate conjugates of compounds of Formula I.

Returning to the scaffold portion of the present compositions, the scaffold comprises hydroxyapatite and tricalcium phosphate in a ratio of from 0/100 to 15/85, collagen, and bioactive glass, wherein the bioactive glass is uniformly dispersed in both interior and surface portions of the scaffold. The scaffold is designed to induce bone formation reliably and reproducibly in a mammalian body and preferably includes particles of porous materials (comprising HA, TCP, collagen and bioactive glass). The scaffold may also have pores, which preferred to be of a dimension to permit progenitor cell migration into the matrix and subsequent differentiation and proliferation. In some embodiments, the pore size of the scaffold is at least 5 μm, e.g., at least 5, 10, 15, 20, 25, 30, 40, 50, 60, 70, 80, 90, 100, 125, 150, 175, 200, 250, 300, 400, 450, 500, 550, 600, 650, 700, 750, 800, 850, 900, 950, 1000, 1200, 1500, 1700, or 2000 μm. The scaffold can be fabricated by close packing the material and molding into a shape spanning the bone defect, or by otherwise structuring as desired a material that is biocompatible, and preferably biodegradable or resorbable in vivo to serve as a “temporary scaffold” and substratum for recruitment of migratory progenitor cells, and as a base for their subsequent anchoring and proliferation. In some embodiments, the scaffold comprises a mesh structure, a foam structure, a sponge structure, or a fiber structure.

As noted above, the scaffold comprises hydroxyapatite and tricalcium phosphate in a ratio of from 0% hydroxyapatite/100% tricalcium phosphate (0/100) to 15% hydroxyapatite (HA)/85% tricalcium phosphate (TCP) (15/85). In some embodiments the ratio of HA to TCP is: 0/100, 1/99, 2/98, 3/97, 4/96, 5/95, 6/94, 7/93, 8/92, 9/91, 10/90, 11/89, 12/88, 13/87, 14/86, or 15/85. In some embodiments the ratio of HA to TCP is: 0/100. In some embodiments the ratio of HA to TCP is: 1/99. In some embodiments the ratio of HA to TCP is: 2/98. In some embodiments the ratio of HA to TCP is: 3/97. In some embodiments the ratio of HA to TCP is: 4/96. In some embodiments the ratio of HA to TCP is: 5/95.

Suitable densities can be from 0.30 to 1.0 g/cc of scaffold, or from 0.4 to 0.8 g/cc of scaffold. In some embodiments, the final scaffold density is about 0.5 to 0.7 g/cc of scaffold.

The amount of HA/TCP present in the compositions can vary, though is generally present in the scaffold composition in an amount of from 200 mg to 1000 mg per cc of scaffold. In some embodiments, the amount of HA/TCP present in the scaffold composition is from 250 mg to 750 mg per cc of scaffold. In some embodiments, the amount of HA/TCP present in the scaffold composition is from 300 mg to 700 mg per cc of scaffold. In some embodiments, the amount of HA/TCP present in the scaffold composition is from 320 mg to 650 mg per cc of scaffold. In still other embodiments, the amount of HA/TCP present in the scaffold composition is from 318 mg to 636 mg per cc of scaffold. In some embodiments, the amount of HA/TCP is present in the scaffold composition is about 323 mg per cc of scaffold.

Collagen is another component of the present compositions and can be obtained from a variety of commercial sources. In general, a collagen 1 binder is useful in the present compositions. In some embodiments the collagen will be crosslinked. In some embodiments, the collagen is crosslinked before addition of bioactive glass. Type I collagen is the most abundant collagen of the human body, forming large, eosinophilic fibers known as collagen fibers. It is present in a variety of tissues, tendons, ligaments, the endomysium of myofibrils, and the organic part of bone, among others. The amount of collagen present in the compositions can vary, though is generally present in the scaffold composition in an amount of from 0 to 50 mg per cc of scaffold. In some embodiments, the amount of collagen present in the scaffold composition is from 7 to 34 mg per cc of scaffold. In some embodiments, the amount of collagen is present in the scaffold composition is about 12 mg to 22 mg per cc of scaffold. In some embodiments, the amount of collagen is present in the scaffold composition is about 22 mg per cc of scaffold

Bioactive glass is yet another component of the present compositions and can also be obtained from a variety of commercial sources. Bioactive glass can be used having a particle size from about 50 to about 500 microns. In some embodiments, the bioactive glass has a particle size from 50 to 200 microns, from 75 to 150 microns, from 100 to 300 microns or from 90 to 250 microns. The amount of bioactive glass in the present compositions can vary, though is generally present in the scaffold composition in an amount of from 20 to 300 mg per cc of scaffold. In some embodiments, the amount of bioactive glass is present in the scaffold composition in an amount of from 20 to 200 mg per cc of scaffold. In some embodiments, the amount of bioactive glass is present in the scaffold composition in an amount of from 75 to 150 mg per cc of scaffold. In some embodiments, the amount of bioactive glass is present in the scaffold composition in an amount of about 125 mg per cc of scaffold.

Bioglass 45S5 or calcium sodium phosphosilicate, a bioactive glass specifically composed of 43-47 wt % SiO₂, 22.5-26.5 wt % CaO, 22.5-26.5 wt % Na2O, and 5-7 wt % P₂O₅.

Other bioactive glasses include S53P4: 53 wt % SiO₂, 23 wt % Na2O, 20 wt % CaO and 4 wt % P2O5; 58S: 58 wt % SiO₂, 33 wt % CaO and 9 wt % P2O5; 70S30C: 70 wt % SiO₂, 30 wt % CaO; 13-93: 53 wt % SiO₂, 6 wt % Na2O, 12 wt % K₂O, 5 wt % MgO, 20 wt % CaO, 4 wt % P₂O₅. In general, the bioactive glasses have compositions within the following wt % ranges: 43-70% SiO₂, 20-30% CaO, 0-26.5% Na2O, 0-9% P₂O₅, 0-12% K₂O, 0-5% MgO.

The compounds of formula I can be added to the scaffold as a neat solid form, or in solution (e.g., a saline solution or water then allowing the compound to crystallize within and on the scaffold). Compound crystals can be but are not limited to amorphous, cuboidal, or needle crystals. Compound crystal dissolution in cell media or biological fluids occurs slowly over time to provide a slow and consistent release of compound into solution or surrounding bone tissue that is capable of inhibiting sclerostin. Compound crystals are produced by taking solid compound into water or saline with agitation (i.e. sonication) to give, for example, a 1.8 mM solution. For each cc of scaffold+bioactive glass, 10-2000 uL 1.8 mM bone-forming agent (API) is added to diffuse upon and throughout the scaffold or device material. The compound can then be allowed to crystallize and dry within scaffold over 24 hours or longer (depending on overall size and thickness of scaffold material). Alternatively, compound crystals can also be formed independently and do not require scaffold material to form.

The scaffold portion of the present compositions can further comprise a synthetic, a biologic material, or a combination thereof. In some embodiments, the scaffold or matrix comprises a naturally occurring polymer, a synthetic biodegradable polymer, a synthetic nonbiodegradable polymer, a bioceramic, a bioactive glass, or combinations thereof. Natural and synthetic polymers, bioceramics, and bioactive glasses for use in scaffolds are known in the art. See, e.g., Dhandayuthapani et al., International Journal of Polymer Science, volume 2011, article ID 290602 (2011), incorporated by reference herein. Natural polymers include, but are not limited to, proteins (e.g., silk, collagen, gelatin, fibrinogen, elastin, keratin, actin, and myosin), polysaccharides (e.g., cellulose, amylose, dextran, chitin, chitosan, and glycosaminoglycans), and polynucleotides (e.g., DNA and RNA). Synthetic polymers include, but are not limited to, PLA, PGA, PLLA, PLGA, PCL, PLDLA, PDS, PGCL, PEA, PCA, PDLLA, PEU, and PBT. Bioceramics and bioactive glasses include, but are not limited to, HAP, TCP, CP ceramics, BCP, and TCP. In some embodiments, the scaffold or matrix is a hydrogel scaffold, a fibrous scaffold, a microsphere scaffold, a polymer-bioceramic composite scaffold, or an acellular scaffold.

In some embodiments, the scaffold or matrix is an osteoconductive matrix. Non-limiting examples of suitable additional osteoconductive matrix materials include, for example, homopolymers or copolymers of glycolic acid, lactic acid, and butyric acid, including derivatives thereof; and ceramics, biphasic calcium phosphate and other calcium phosphates, and calcium sulphates, or combinations thereof typically, osteoconductive matricies contemplated herein include at least one of the previously listed materials. Other additional matrix/scaffold components useful in the present compositions include, but are not limited to, biocomposite bone grafts, Kryptonite bone cement (Doctors Research Group, Oxford, CT), Vitoss, Vitoss BA, Orthoblend, Grafton, Arthrex, Allograft, Cadaverbone, Ostoset, Novabone, Augmatrix, Mastergraft, Hydroset, Pro-dense, Pro-stim, hydroset, (porous) tantalum bone graft, titanium mesh, titanium bone graft, and Genex bone graft. Combinations of these matrix materials also can be useful. The osteoconductive matrix components can also include a further structural support such as a calcium salt, calcium sulfate, calcium phosphate, a calcium phosphate cement, dicalcium phosphate, calcium carbonate, collagen, plaster of Paris, phosphophoryn, a borosilicate, a biocompatible ceramic, a calcium phosphate ceramic, polytetrafluoroethylene, sulfate salt, homopolymers or copolymers of glycolic acid, lactic acid, and butyric acid, including derivatives thereof; and ceramics, biphasic calcium phosphate and other calcium phosphates, and calcium sulphates.

In some embodiments, the osteoconductive matrix comprises an osteoinductive agent and, optionally, a structural support. The osteoinductive agent can be any agent that promotes bone formation. In some embodiments, the osteoinductive agent is bone allograft, bone autograft, bone marrow aspirate, demineralized bone, or periodontal ligament cells.

A variety of sterilization techniques can be used in the preparation of the final compositions, prior to packaging. These include, but are not limited to gamma irradiation, E-beam irradiation, UV irradiation, steam heat, dry heat, plasma, Et₂O, PAA, ethanol, and/or iodine.

In another aspect, provided herein are compositions for bone formation, comprising a scaffold of hydroxyapatite (HA) and tricalcium phosphate (TCP) in a ratio of from 0/100 to 15/85, collagen, and bioactive glass, wherein the bioactive glass is uniformly dispersed in both interior and surface portions of the scaffold, and wherein the composition is sterilized and packaged.

As in the related sterilized compositions above, this group of compositions are drawn to structural (and non-structural) scaffolds prepared from combinations of hydroxyapatite (HA), tricalcium phosphate (TCP), collagen, and bioactive glass. In some embodiments, the ratio of HA to TCP is 0/100, 1/99, 2/98, 3/97, 4/96, 5/95, 6/94, 7/93, 8/92, 9/91, 10/90, 11/89, 12/88, 13/87, 14/86 or 15/85.

Generally, the compositions can be prepared using commercial sources of HA and TCP, with added collagen (obtainable from a variety of sources). Bioactive glass can then be added to the HA/TCP/collagen scaffold by a variety of techniques to uniformly disperse the bioactive glass in both interior and surface portions of the scaffold.

For any of the compositions described herein, the sterilized product can be in a form which is moldable, structurally rigid, or in the form of a putty or liquid cement.

### A. Additional Bone-Forming Agents

For any of the embodiments described above, an additional bone-forming agent can be used, either in place of the compounds of formula (I), or in addition to those compounds. Moreover, any of the bone-forming agents described herein can be added to the compositions of Embodiment 2 (below).

Suitable bone-forming agents are described in, for example, WO2020/037001, WO2020/037004, and WO2014/153203.

In some embodiments, the bone-forming agents are represented by the formula:

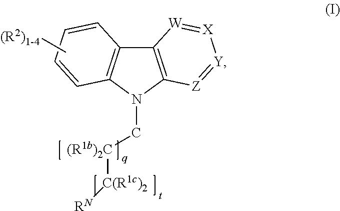

or a salt, hydrate, or isomer thereof, wherein:
W is selected from CR³a and N, wherein N is optionally oxidized to the corresponding N-oxide;
X is selected from CR^{3b} and N, wherein N is optionally oxidized to the corresponding N-oxide;
Y is selected from CR^{3c} and N, wherein N is optionally oxidized to the corresponding N-oxide;
Z is selected from CR^{3d} and N, wherein N is optionally oxidized to the corresponding N-oxide;
R^{N} is selected from the group consisting of NR⁶R⁷, heterocyclyl, and heteroaryl, wherein heterocyclyl and heteroaryl comprise from about 5 to about 10 ring atoms, at least one of which is nitrogen, and wherein any N in R^{N} is optionally oxidized to the corresponding N-oxide; each R^{1a}, R^{1b}, and R^{1c} is independently selected from H, methyl, and ethyl, wherein the total number of carbon atoms in the group —C(R^{1a})₂—[C(R^{1b})₂]_{q}—[C(R^{1c})₂]ₜ— does not exceed six; each R², R^{3a}, R^{3b}, R^{3c} and R^{3d} is independently selected from the group consisting of H, halogen, C₁₋₆ alkyl, C₁₋₆ haloalkyl, C₂₋₆ alkenyl, C₂₋₆ alkynyl, C₁₋₆ alkoxy, C₁₋₆ haloalkoxy, aryloxy, C₁₋₆ alkyl-OH, —OR⁴, —C₀₋₆ alkyl-NR⁴R⁵, —SR⁴, —C(O)R⁴, —C₀₋₆ alkyl-C(O)OR⁴, —C(O)NR⁴R⁵, —N(R⁴)C(O)R⁵, —N(R⁴)C(O)OR⁵, —N(R⁴)C(O)NR⁴R⁵, —OP(O)(OR⁴)₂, —S(O)₂OR⁴, —S(O)₂NR⁴R⁵, —CN, cycloalkyl, heterocycloalkyl, aryl and heteroaryl;
alternatively, two R² groups on adjacent atoms can be combined with the atoms to which they are attached to form a member selected from the group consisting of cycloalkyl, heterocycloalkyl, aryl and heteroaryl;
each R⁴, R⁵, R⁶, and R⁷ is independently selected from the group consisting of H, C₁₋₆ alkyl, and C₁₋₆ alkyl-OH;
the subscript q is an integer from 0 to 4; and
the subscript t is an integer from 0 to 4;
provided that no more than one of W, X, Y, and Z is N or the corresponding N-oxide;
provided that when:
a) the sum of q and t is 1, and
b) either of R⁶ or R⁷, if present, is H or C₁₋₆ alkyl,
at least one of R^{1a} and R^{1b} is other than H; and
provided that when the sum of q and t is 2,
a) R² is other than H, and
b) at least one of R⁶ and R⁷, if present, is other than H or methyl.

In other embodiments, the bone-forming agents are represented by the formula:

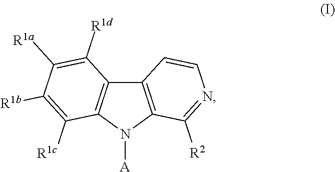

or a salt or hydrate thereof; wherein
R^{1b} is C₁₋₆ alkoxy;
R² selected from the group consisting of H, C₁₋₆ alkyl, and C₁₋₆ haloalkyl;
each R^{1a}, R^{1b}, and R^{1d} is independently H or halogen,
provided that no more than two of R^{1a}, R^{1c}, and R^{1d} is H;
   A is

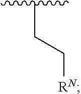

R^{N} is selected from the group consisting of heterocyclyl and heteroaryl, wherein
the heterocyclyl moiety is selected from monocyclic, fused bicyclic, and bridged cyclic, the monocyclic heterocyclyl comprising from 4 to 7 ring members, the fused bicyclic and bridged bicyclic heterocyclyl comprising from 7 to 10 ring members, each heterocyclyl moiety having from 1 to 3 heteroatoms as ring members selected from N, O, and S, wherein each heterocyclyl moiety comprises at least one nitrogen atom as a ring member and is optionally substituted with from 1 to 3 R⁵ moieties,
the heteroaryl moiety comprises from 5 to 10 ring members, wherein at least one ring member is a nitrogen atom and is optionally substituted with from 1 to 3 R⁵ moieties; and
each R⁵ is selected from the group consisting of —OH, C₁₋₃ alkyl, C₁₋₃ alkyl-OH, —O—C₁₋₃ alkyl, C₃₋₄ heteroalkyl, C₁₋₃ haloalkyl, —O—C₁₋₃ haloalkyl, halogen, and oxo.

In some embodiments, the bone-forming agent is selected from the group consisting of:

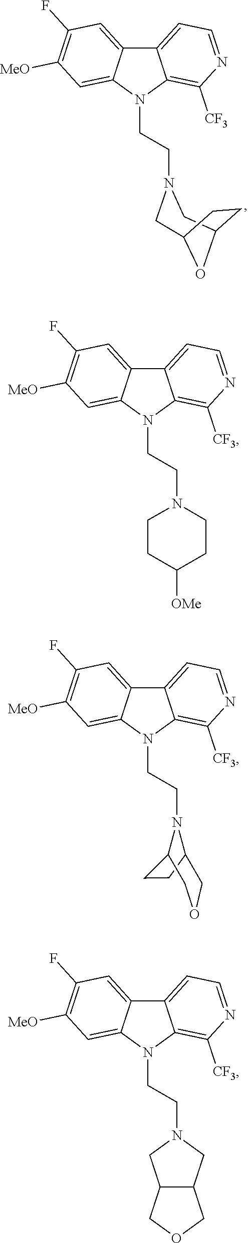 [SMILES: COc1cc2c(cc1F)c1ccnc(C(F)(F)F)c1n2CCN1CC2CCC(C1)O2 ; COc1cc2c(cc1F)c1ccnc(C(F)(F)F)c1n2CCN1CCC(OC)CC1 ; COc1cc2c(cc1F)c1ccnc(C(F)(F)F)c1n2CCN1C2CCC1COC2 ; COc1cc2c(cc1F)c1ccnc(C(F)(F)F)c1n2CCN1CC2COCC2C1]

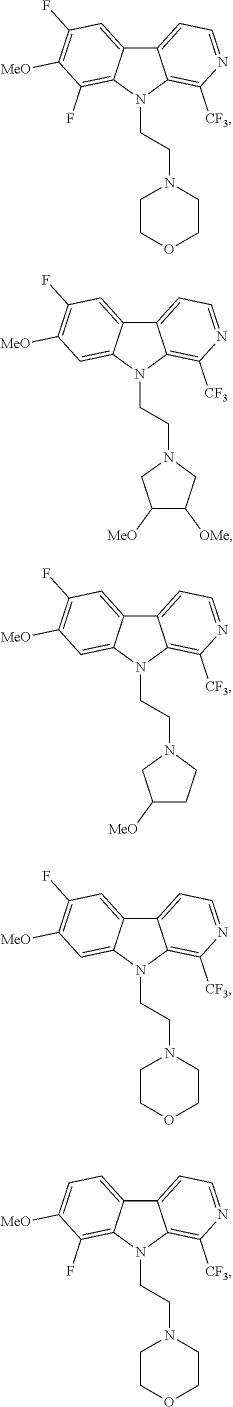 [SMILES: COc1c(F)cc2c3ccnc(C(F)(F)F)c3n(CCN3CCOCC3)c2c1F ; COc1cc2c(cc1F)c1ccnc(C(F)(F)F)c1n2CCN1CC(OC)C(OC)C1 ; COc1cc2c(cc1F)c1ccnc(C(F)(F)F)c1n2CCN1CCC(OC)C1 ; COc1cc2c(cc1F)c1ccnc(C(F)(F)F)c1n2CCN1CCOCC1 ; COc1ccc2c3ccnc(C(F)(F)F)c3n(CCN3CCOCC3)c2c1F]

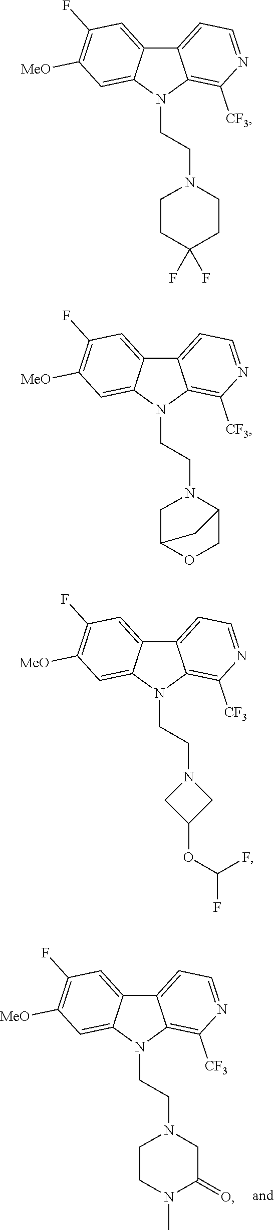 [SMILES: COc1cc2c(cc1F)c1ccnc(C(F)(F)F)c1n2CCN1CCC(F)(F)CC1 ; COc1cc2c(cc1F)c1ccnc(C(F)(F)F)c1n2CCN1CC2CC1CO2 ; COc1cc2c(cc1F)c1ccnc(C(F)(F)F)c1n2CCN1CC(OC(F)F)C1 ; COc1cc2c(cc1F)c1ccnc(C(F)(F)F)c1n2CCN1CCN(C)C(=O)C1]

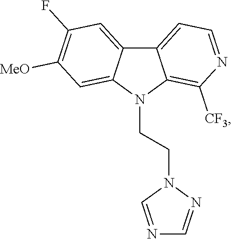 [SMILES: COc1cc2c(cc1F)c1ccnc(C(F)(F)F)c1n2CCn1cncn1]

or a salt or hydrate thereof.

### IV. Methods of Promoting Bone Formation

In another aspect, the present invention provides a method of promoting bone formation and fusion in a subject in need thereof, by locally administering to the subject a therapeutically effective amount of a composition of the present invention (e.g., a composition comprising a scaffold as described above, optionally with a composition described herein.

One of skill in the art will appreciate that osteoblast mineral deposit (bone formation) can be achieved by local, systemic, or both local and systemic administration. In some embodiments, bone formation is local. A subject in need of local bone formation may have any of a variety of ailments or disease states (including but not limited to, weakened bone, fractured bone, or a disease or condition characterized by low bone mass or poor mineralization as described herein). In some embodiments, the subject is in need of a spinal fusion, bone fusion, arthrodesis, or an orthopedic, dental, or periodontal synthetic bone graft or implant. In some embodiments, the present invention provides a method of promoting bone formation at a site of injury or localized condition. In some embodiments, the present invention comprises a method of fusing bones (e.g., at a site of injury). In some embodiments, the site of injury is a surgical site. In other embodiments, the injury is a fracture or weakened bone or periodontal disease.

In some embodiments, bone formation is systemic. Systemic bone formation refers to the formation of bone throughout the subject, and can affect all the bones in the subject's body. A subject in need of systemic bone formation can suffer from any of a variety of ailments or disease states. In some embodiments, the subject suffers from a low bone mass/density condition/disease (either primary or secondary), a bone fracture, a periodontal disease/condition, or a disease/condition causing poor bone mineralization (e.g., ostoegenesis imperfect or HPP). Low bone mass can be determined by a variety of methods known to one of skill in the art. For example, low bone mass/density can be characterized by a T-score less than about −0.5. Low bone mass/density diseases/conditions include, but are not limited to, osteoporosis, osteopenia, and osteoporosispseudoglioma syndrome (OPPG), glucocorticoid induced low bone mass/density, Osteogenesis imperfecta. In some other embodiments, the low bone mass condition/disease can be osteopenia or osteoporosispseudoglioma syndrome (OPPG), HPP, or glycocorticoid induced low bone mass/density or other diseases which result in secondary low bone density conditions.

Local and/or systemic bone formation using a compound or composition of the present invention can be achieved according to any of a variety of methods. Methods of formulating and administering the compounds and compositions of the present invention (e.g., a compound or composition of Formula I) are described in Section VII below. In some embodiments, the method of promoting bone formation comprises implanting a medical device as described herein (e.g., in Section VIII below) to subject in need thereof.

The methods of promoting osteoblast mineral deposits, ultimately increasing bone mineralization or density, can be used to treat patients with diseases characterized by secondary induced osteoporosis (low bone mass) including, but not limited to, osteomalacia, Polyostotic fibrous dysplasia, osteogenesis imperfecta, Paget's disease, rheumatoid arthritis, zero gravity, osteoarthritis, Prolonged inactivity or immobility, arthrodesis, osteomyelitis, Celiac disease, Crohn's Disease, Ulcerative Colitis, inflammatory bowel disease, gastrectomy, secondary induced osteoporosis, Amennorhea, Cushing's Disease, Cushing's syndrome, Diabetes Mellitus, Diabetes, Eating Disorders, Hyperparathyroidism, Hyperthyroidism, Hyperphosphatasia (HPP), Hyperprolactinemia, Kleinefelter Syndrome, Thyroid Disease, Turner Syndrome, steroid induced osteoporosis, seizure or depression induced osteoporosis, immobility, arthritis, cancer induced secondary osteoporosis, Gonadotropin releasing hormone agonists induced low bone mass, Thyroid medication induced low bone mass, Dilantin (phenytoin), depakote induced low bone mass, chemotherapy induced low bone mass, Immunosuppressant induced low bone mass, Blood thinning agents induced low bone mass, Grave's disease, Juvenile rheumatoid arthritis, Malabsorption syndromes, Anorexia nervosa, Kidney disease, Anticonvulsant treatment (e.g., for epilepsy), Corticosteroid treatment (e.g., for rheumatoid arthritis, asthma), Immunosuppressive treatment (e.g., for cancer), Inadequate nutrition (especially calcium, vitamin D), Excessive exercise leading to amenorrhea (absence of periods), Smoking, and Alcohol abuse, pregnancy-associated osteoporosis, copper deficiency, Dibasic aminoaciduria type 2, Werner's syndrome, Hajdu-Cheney syndrome, Hyperostosis corticalis deformans juvenilis, Methylmalonic aciduria type 2, Cystathionine beta-synthase deficiency, Exemestane, Hyperimmunoglobulin E (IgE) syndrome, Haemochromatosis, Singleton-Merten syndrome, Beta thalassaemia (homozygous), Reflex sympathetic osteodystrophy, Sarcoidosis, Winchester syndrome, Hallermann-Streiff syndrome (HSS), Cyproterone, Glycerol kinase deficiency, Bonnet-Dechaume-Blanc syndrome, Prednisolone, Heparin, Geroderma osteodysplastica, Torg osteolysis syndrome, Orchidectomy, Fabry's disease, Pseudoprogeria syndrome, Wolcott-Rallison syndrome, Ankylosing spondylitis, Myeloma, Systemic infantile hyalinosis, Albright's hereditary osteodystrophy, Anorexia Nervosa, Autoimmune Lymphoproliferative Syndrome, Brown-Sequard Syndrome, Diamond-Blackfan anemia, Eating disorders, Galactorrhoea-Hyperprolactinaemia, Gonadal dysgenesis, Kidney conditions, Menkes Disease, Menopause, Neuritis, Ovarian insufficiency due to FSH resistance, Familial Ovarian insufficiency, Premature aging, Primary biliary cirrhosis, Prolactinoma, Familial Prolactinoma, Renal osteodystrophy, Ulcerative colitis, Underweight, Werner syndrome, Bone tumor, Bone cancer, Brittle bone disease, Osteonecrosis, Osteogenesis imperfecta congenita, Osteogenesis imperfecta tarda, osteogenesis imperfecta, glucocorticoid induced osteopenia/osteoporosis and periodontal disease. One of skill in the art will appreciate that other types of conditions, diseases and treatments also lead to osteoporosis.

Bone formation can be measured according to any of a variety of ways known to one of skill in the art. Methods of measuring bone formation include, but are not limited to, uCT (micro CT), Dual X-ray absorption (Bone density), ultrasound, QCT, SPA, DPA, DXR, SEXA, QUS, X-ray, using the human eye during surgically manipulation, Alizarin red S, serum osteocalcin, serum alkaline phosphatase, Serum bone Gla-protein (BGP), bone mineral content, bone ash weight, serum calcium, serum phosphorus, tantalum markers, and serum IGF-1.

Many indicators of bone formation can be used to measure and/or quantify the amount of bone formation, including bone density. In some embodiments, bone formation can be demonstrated by an increase of 0.1% in bone density. In other embodiments, bone growth can be demonstrated by an increase of 0.2%, 0.3%, 0.4%, 0.5%, 0.6%, 0.7%, 0.8%, 0.9%, 1%, 2%, 3%, 4%, 5%, 6%, 7%, 8%, 9%, 1, 12%, 14%, 16%, 18%, 20%, 30%, 40%, 50%, 60%, 70%, 80%, 90%, 100%, 200%, 300%, 400%, 500%, 600%, 700%, 800%, 900% or 1000% or greater, in bone density. Bone density can be measured by a variety of different methods, including the T-score and Z-score. The Z-score is the number of standard deviations above or below the mean for the patient's age and sex. The T-score is the number of standard deviations above or below the mean for a healthy 30 year old adult of the same sex as the patient. Low bone mass is characterized by a T-score of −1 to −2.5. Osteoporosis is characterized by a T-score less than −2.5. Improvement in the T-score or Z-score indicate bone growth. Bone density can be measured in a variety of places of the skeleton, such the spine or the hip. One of skill in the art will appreciate that other methods of determining bone density are useful in the present invention.

### V. Methods of Treating Bone Loss

In another aspect, the present invention provides a method of treating bone loss by administering locally to a subject suffering from bone loss, a therapeutically effective amount of a composition of the present invention (e.g., a scaffold or composition, optionally comprising a composition described herein, or another bone forming agent as described herein).

Bone density in an individual can be described by the net loss (bone resorption) and gain (bone formation) of bone mass. In individuals with bone loss, the net bone resorption is greater than the bone formation causing a decrease in bone. It is contemplated that in particular embodiments of this invention, bone loss may be treated by inhibiting or reducing bone resorption while stimulating or encouraging bone formation.

Antiresorptive agents are compounds which slow the process of bone resorption. Antiresorptive agents include, but are not limited to, RankL inhibitors, Denosumab, Prolia, Cathepsin-K modulators, Alendronate, Fosamax, selective estrogen receptor modulators (SERMS), Calcium, Estrogen, Bisphosphonates, and Calcitonin.

The compositions of the present invention treat bone loss by promoting bone formation. When the compounds of the present invention are administered sequentially or combination with one or more antiresorptive agents, both the rate of bone resorption is inhibited or reduced and the rate of bone formation is stimulated.

The compositions of the present invention and the antiresorptive agents described herein may be administered sequentially or in combination.

The compositions of the present invention treat bone loss by promoting bone formation. In some embodiments, when the compounds of the present invention are administered sequentially with one or more antiresorptive agents, the rate of bone resorption is inhibited or reduced and the amount of bone formation is maintained.

The compositions of the present invention may be administered to patients who have been treated with an antiresorptive, thus serially, or patients may be administered sequentially with one or more antiresorptive agents, whereby the rate of bone resorption is inhibited or reduced and the amount of bone formation is maintained.

Subjects to be treated with the compositions and methods of the present invention can be any mammal, for example, a human or a non-human mammal, e.g., a primate, dog, cat, horse, cow, goat, sheep, pig, mouse, or rat, or any commercially important animal or domesticated animal.

In some embodiments, a subject to be treated according to the methods of the present invention is an individual who has received or is receiving an antiresorptive therapeutic agent.

For example, in some embodiments, antiresorptive therapy may be administered concurrently with a composition of the present invention. In some embodiments, antiresorptive therapy and therapy with a composition of the present invention are administered sequentially (either antiresorptive therapy preceding therapy with a composition of the present invention, or therapy with a composition of the present invention preceding antiresorptive therapy). In some embodiments, the individual may have been previously treated with an antiresorptive agent. In some embodiments, an individual to be treated according to the methods of the present invention has not been treated with an antiresorptive agent. In some embodiments, an individual is treated with an antiresorptive agent after being treated with a composition of the present invention.

In some embodiments, an individual to be treated according to the methods of the present invention is an individual who has received or is receiving a combination of antiresorptive and/or bone anabolic therapeutic agents. For example, in some embodiments, antiresorptive and/or bone anabolic therapy may be administered concurrently with a composition of the present invention. In some embodiments, antiresorptive and/or bone anabolic therapy and therapy with a composition of the present invention are administered sequentially (either antiresorptive therapy preceding therapy with a composition of the present invention, or therapy with a composition of the present invention preceding antiresorptive therapy). In some embodiments, the individual may have been previously treated with an antiresorptive and/or bone anabolic agent. In some embodiments, an individual may be concurrently treated with an antiresorptive and/or bone anabolic agent during a first portion of the treatment course for the compound or composition of the present invention but may discontinue treatment with the antiresorptive and/or bone anabolic agent during a second portion of the treatment course. In some embodiments, an individual to be treated according to the methods of the present invention has not been treated with an antiresorptive agent and/or bone anabolic. In some embodiments, an individual is treated with an antiresorptive and/or bone anabolic agent after being treated with a composition of the present invention.

In some embodiments, the compositions of the present invention are administered systemically. In some embodiments, the compounds and compositions of the present invention are administered locally.

The compounds of the present invention may also be included in slow release formulations for prolonged treatment following a single dose. In one embodiment, the formulation is prepared in the form of microspheres. The nanoparticle/microspheres can be prepared as a homogenous matrix of a compound with a biodegradable controlled release material, with optional additional medicaments as the treatment requires. The nanoparticle/microspheres are preferably prepared in sizes suitable for infiltration and/or injection, and injected systemically, or directly at the site of treatment.

Some slow release embodiments include polymeric substances that are biodegradable and/or dissolve slowly. Such polymeric substances include polyvinylpyrrolidone, low- and medium-molecular weight hydroxypropyl cellulose and hydroxypropyl methylcellulose, crosslinked sodium carboxymethylcellulose, carboxymethyl starch, collagen, potassium methacrylate divinylbenzene copolymer, polyvinyl alcohols, starches, starch derivatives, microcrystalline cellulose, ethylcellulose, methylcellulose, and cellulose derivatives, β-cyclodextrin, captisol, poly(methyl vinyl ethers/maleic anhydride), glucans, scierozlucans, mannans, xanthans, alzinic acid and derivatives thereof, dextrin derivatives, glyceryl monostearate, semisynthetic glycerides, glyceryl palmitostearate, glyceryl behenate, polyvinylpyrrolidone, gelatine, magnesium stearate, stearic acid, sodium stearate, talc, sodium benzoate, boric acid, and colloidal silica.

Slow release agents of the invention may also include adjuvants such as starch, pregelled starch, calcium phosphate mannitol, lactose, saccharose, glucose, sorbitol, microcrystalline cellulose, gelatin, polyvinylpyrrolidone. methylcellulose, starch solution, ethylcellulose, arabic gum, tragacanth gum, magnesium stearate, stearic acid, colloidal silica, glyceryl monostearate, hydrogenated castor oil, waxes, and mono-, bi-, and tri-substituted glycerides. Slow release agents may also be prepared as generally described in WO94/06416.

### A. Local Delivery

The compositions of the present invention are designed to be administered locally. Local administration of the compositions of the present invention can be used, for example, for fracture healing, fusion (e.g., arthrodesis), orthopedic reconstruction, and periodontal repair. In some embodiments, local administration comprises administering a composition in conjunction with a second material capable of maintaining the composition at an in vivo site of application or capable of providing structural load. In some embodiments, the second material is biocompatible, a matrix, in vivo biodegradable or resorbable, and/or porous enough to allow cell infiltration. In some embodiments, a composition of the present invention (e.g., a composition including a compound of Formula I) is administered locally into a surgical site and/or with an implantable medical device.

A scaffold or matrix for use in delivering a compound of the present invention can comprise a synthetic, a biologic material, or a combination thereof. In some embodiments, the scaffold or matrix comprises a naturally occurring polymer, a synthetic biodegradable polymer, a synthetic nonbiodegradable polymer, a bioceramic, a bioactive glass, or combinations thereof. Natural and synthetic polymers, bioceramics, and bioactive glasses for use in scaffolds are known in the art. See, e.g., Dhandayuthapani et al., International Journal of Polymer Science, volume 2011, article ID 290602 (2011), incorporated by reference herein. Natural polymers include, but are not limited to, proteins (e.g., silk, collagen, gelatin, fibrinogen, elastin, keratin, actin, and myosin), polysaccharides (e.g., cellulose, amylose, dextran, chitin, chitosan, and glycosaminoglycans), and polynucleotides (e.g., DNA and RNA). Synthetic polymers include, but are not limited to, PLA, PGA, PLLA, PLGA, PCL, PLDLA, PDS, PGCL, PEA, PCA, PDLLA, PEU, and PBT. Bioceramics and bioactive glasses include, but are not limited to, HAP, TCP, CP ceramics, BCP, and TCP and 45S5. In some embodiments, the scaffold or matrix is a hydrogel scaffold, a fibrous scaffold, a microsphere scaffold, a polymer-bioceramic composite scaffold, or an acellular scaffold.

In some embodiments, the osteoconductive matrix comprises an osteoinductive agent and, optionally, a structural support. The osteoinductive agent can be any agent that promotes bone formation. In some embodiments, the osteoinductive agent is bone allograft, bone autograft, demineralized bone, bone marrow aspirate or periodontal ligament cells.

### B. Combination Therapy

In practicing the methods of the present invention, the pharmaceutical compositions can be used alone, or in combination with other therapeutic or diagnostic agents. Additionally, the medical devices described herein include the use of the compositions described herein alone or in combination with an other therapeutic or diagnostic agents. The additional drugs used in the combination protocols of the present invention can be administered separately or one or more of the drugs used in the combination protocols can be administered together, such as in an admixture. Where one or more drugs are administered separately, the timing and schedule of administration of each drug can vary. The other therapeutic or diagnostic agents can be administered at the same time as the compounds of the present invention, separately or at different times.

In some embodiments, a compound or composition as described herein (e.g., a composition described herein) is administered in combination with one or more other therapeutic agents. When a compound of the present invention and is combined with another agent, the two can be co-administered or administered separately. Co-administration includes administering the other agent within 0.5, 1, 2, 4, 6, 8, 10, 12, 16, 20, or 24 hours, as well as within 1 to 7 days (e.g., 1, 2, 3, 4, 5, 6, or, 7 days), 1 to 4 weeks (e.g., 1, 2, 3, or 4 weeks), or 1 or 18 months (e.g., 1, 2, 3, 4, 5, 6, 7, 8, 9, 10, 11, 12, 13, 14, 15, 16, 17, or 18 months) of administering the compound of the present invention. Co-administration also includes administering the other agent and the compound of the present invention simultaneously, approximately simultaneously (e.g., within about 1, 5, 10, 15, 20, or 30 minutes, or on the same day, or on the same week, or on the same month of each other), or sequentially in any order. In some embodiments, co-administration comprises administering another agent (e.g., an antiresorptive) for a period of time (e.g., weeks, months, or years), then administering a composition described herein (e.g., days, weeks, months, or years), then administering the other agent (e.g., antiresorptive) either alone or in combination with the compositions described herein. In some embodiments, the other agent and the compound of the present invention can each be administered once a day, or two, three, or more times per day so as to provide the preferred dosage level per day.

In some embodiments, co-administration can be accomplished by co-formulation, i.e., preparing a single pharmaceutical composition including both a compound of the present invention and the second therapeutic agent (e.g., the antiresorptive agent). In other embodiments, the compound of the present invention and the second therapeutic agent are formulated separately.

The one or more other therapeutic agents can be delivered by any suitable means. The pharmaceutical preparation is preferably in unit dosage form. In such form the preparation is subdivided into unit doses containing appropriate quantities of the antiresorptive agent and/or the compound of the present invention. The unit dosage form can be a packaged preparation, the package containing discrete quantities of preparation, such as packeted tablets, capsules, and powders in vials or ampoules. Also, the unit dosage form can be a capsule, tablet, cachet, patch, or lozenge itself, or it can be the appropriate number of any of these in packaged form.

The one or more other therapeutic agents can be present in any suitable amount, and can depend on various factors including, but not limited to, weight and age of the subject, state of the disease, etc. Suitable dosage ranges for the one or more other therapeutic agents in combination with the a compound or composition of the present invention include from about about 0.0001 ug to about 10,000 mg, or about 0.0001 ug to about 1000 mg, or about 0.0001 ug to about 500 mg, or about 0.0001 ug to about 1000 ug, 0.1 ug to about 10,000 mg, or about 0.1 ug to about 1000 mg, or about 0.1 ug to about 500 mg, or about 0.1 ug to about 1000 ug or about 1 ug to about 1000 mg, or about 1 ug to about 500 mg, or about 1 ug to about 50 mg, or about 1 ug to about 1000 ug, or about 10 ug to about 1000 mg, or about 10 ug to about 500 mg, or about 10 ug to about 50 mg, or about 0.1 mg to about 10,000 mg, or about 1 mg to about 1000 mg, or about 10 mg to about 750 mg, or about 25 mg to about 500 mg, or about 50 mg to about 250 mg. Suitable dosages for the one or more other therapeutic agents in combination with a compound or composition of the present invention, include about 0.01, 0.1, 1, 5, 10, 20, 30, 40, 50, 60, 70, 80, 90, 100, 200, 300, 400, 500, 600, 700, 800, 900, 1000, 1250, 1500, 1750 or 2000 mg.

The one or more other therapeutic agents and the compound or composition of the present invention can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w) or about 1:1 (w/w). Other dosages and dosage ratios of the antiresorptive agent and the compound of the present invention are suitable in the compositions and methods of the present invention.

The composition can also contain other compatible therapeutic agents. The compounds described herein can be used in combination with one another, with other active agents, or with adjunctive agents that may not be effective alone, but may contribute to the efficacy of the active agent.

In some embodiments, an individual to be treated according to a method of the present invention is administered a composition as described herein in combination or sequentially with an antiresorptive drug. Antiresorptive drugs include those that slow or block the resorption of bone. Administration of a compound or composition as described herein and an antiresorptive drug can promote local bone growth and/or systemic bone growth. In some embodiments, the administration of a compound compound or composition as described herein and an antiresorptive drug promotes systemic bone growth. Bone growth can be achieved by increasing bone mineral content, increasing bone density and/or growth of new bone. In other embodiments, local application of the compound or composition as described herein and an antiresorptive drug achieves systemic bone growth.

Antiresorptive drugs useful in the methods of the present invention include, but are not limited to, denosumab, Prolia, a RankL inhibitor, a bisphosphonate (e.g., Fosamax, denosumab, Prolia, Actonel, or Reclast, Alendronate, Bonviva™, Zometa™, olpadronate, neridronate, skelid, bonefos), a selective estrogen receptor modulator (SERM) or analog (e.g., Evista), calcitonin, a calcitonin analog (e.g., Miacalcic), parathyroid hormone, calcilytics, calcimimetics (e.g., cinacalcet), statins, anabolic steroids, lanthanum and strontium salts, and sodium fluoride, Vitamin D or a Vitamin D analog, CatK inhibitor, prostaglandin inhibitor, or phosphodiesterase inhibitor type E.

In some embodiments, the antiresorptive drug is denosumab.

Bisphosphonates useful in the methods of the present invention can be any suitable bisphosphonate. In some embodiments, the bisphosphonates are nitrogenous, such as Pamidronate (APD, Aredia), Neridronate, Olpadronate, Alendronate (Fosamax), Ibandronate (Boniva), Risedronate (Actonel) and Zoledronate (Zometa). In other embodiments, the bisphosphonates are non-nitrogenous, such as Etidronate (Didronel), Clodronate (Bonefos, Loron) and Tiludronate (Skelid). One of skill in the art will appreciate that other bisphosphonates are useful in the present invention.

SERMs useful in the methods of the present invention can be any suitable SERM. In some embodiments, the SERM can be clomifene, raloxifene, tamoxifen, toremifene, bazedoxifene, lasofoxifene or ormeloxifene. One of skill in the art will appreciate that other SERMs are useful in the present invention.

The antiresorptive drug can also be any suitable calcitonin analog or cathepsin K inhibitor. In some embodiments, calcitonin analogs useful in the methods of the present invention include, but are not limited to, miacalcic. One of skill in the art will appreciate that other calcitonin analogs are useful in the present invention.

Vitamin D analogs useful in the methods of the present invention can be any suitable Vitamin D analog. In some embodiments, Vitamin D analogs useful in the methods of the present invention include, but are not limited to, Vitamin D1 (molecular compound of ergocalciferol with lumisterol, 1:1), Vitamin D2 (ergocalciferol or calciferol), Vitamin D3 (cholecalciferol), Vitamin D4 (22-dihydroergocalciferol) and Vitamin D5 (sitocalciferol). One of skill in the art will appreciate that other Vitamin D analogs are useful in the present invention.

RankL inhibitors useful in the present invention include any compounds that inhibit the activity of RankL. For example, RankL inhibitors include, but are not limited to, the human monoclonal antibody denosumab or prolia. One of skill in the art will appreciate that other RankL inhibitors are useful in the present invention.

In some embodiments, an individual to be treated according to a method of the present invention is administered a composition as described herein in combination or sequentially with an anabolic agent. Anabolic agents include, but are not limited to, parathyroid hormone (PTH) or an analog thereof, P15, sclerostin inhibitors, sclerostin antibodies, bone morphogenic protein (BMP) or a BMP agonist, a population of bone marrow stem cells, or a population of mesenchymal stem cells.

In some embodiments, the anabolic agent is parathyroid hormone (PTH) or an analog thereof (e.g., teriparatide (Forteo). In some embodiments, the anabolic agent is P15 or an analog thereof. In some embodiments, the anabolic agent is a sclerostin antibody (Mab) inhibitor, which in some embodiments is selected from romosozumab, BPS804, BA058, blosozumab, and AbD09097. In some embodiments, the BMP is selected from the group consisting of BMP2, BMP7, BMP4, BMP5, and BMP6. In some embodiments, the BMP agonist is a compound described in Vrijens K, et al. PLoS One. 2013; 8(3):e59045, the contents of which is incorporated by reference for all purposes. In some embodiments, the anabolic agent is a population of bone marrow stem cells. In some embodiments, the anabolic agent is a population of mesenchymal stem cells.

### VI. Medical Devices

In some embodiments, the present invention provides a medical device formed from a structural support, wherein an implantable portion of the structural support is adapted to be permanently implanted within a subject, wherein the implantable portion is attached to a bone, the structural support bearing at least a partial coating including a composition described herein. In some embodiments, the medical device is an orthopedic or periodontal medical device.

Other aspects of the present invention are directed towards medical implants. Such medical devices and implants include, for example, the osteogenic devices and methods of using the same for repairing endochondral bone and osteochondral defects taught in US patent application publication No. 20060177475 to David Rueger et al., published Aug. 10, 2006, as well as in issued U.S. Pat. Nos. 6,190,880, 5,344,654, 5,324,819, 5,468,845, 6,949,251, 6,426,332 and 5,656,593, and U.S. Publication Nos. 2002/0169122, 2002/0187104, 2006/0252724 and 2007/0172479, the subject matter of which is hereby incorporated by reference.

These medical devices generally provide a structural support having an implantable portion preferentially adapted to mechanically engage bone and/or cartilage as taught, for instance, in U.S. Publication No. 2006/0178752 to Joseph Vaccarino III, et al., published Aug. 10, 2006, the subject matter of which is hereby incorporated by reference. These bone implants desirably comprise an active agent on at least a portion thereof. As shown by U.S. Publication No. 2006/0188542 to John Dennis Bobyn, et al., published Aug. 24, 2006, the subject matter of which is hereby incorporated by reference, the active agent is preferably formulated to be locally deliverable to bone proximate the implant in sustained release or in at least a two-phased release scheme. In the latter, a first phase rapidly releases a first quantity of the active agent, and the second and subsequent phases gradually release a second quantity of the active agent, whereby bone formation stimulated by the active agent is modulated.

Medical devices such as bone implants feature implantable portions bearing a compound or composition of present invention (e.g., a composition described herein) foster quicker and more complete bone formation in situ. The implantable portion of the medical device can be desirable at least partially or totally covered or impregnated with a compound or composition of the present invention. In some embodiments, the medical device is externally coated with a compound or composition as described herein. In some embodiments, the external coating completely coats the implantable portion of the structural support. In some embodiments, the structural support (e.g., matrix or scaffold) comprises a compound or composition as described herein within the support, i.e., internally. In some embodiments, the structural support (e.g., matrix or scaffold) comprises an external coating of a compound or composition as described herein and also comprises the compound or composition within the support, i.e., internally.

Medical devices of the present invention include pins, rods, screws, plates, cages, nails, orthopedic and/or dental implants. In some embodiments, the medical devices are made from material comprising metal, polymer, or ceramic, or from combinations thereof. Metals useful for making medical devices of the present invention include, but are not limited to cobalt, chrome, chromium, stainless steel, titanium, titanium alloys, tantalum, trabecular metal. Polymers useful for making medical devices of the present invention include, but are not limited to ultra high molecular weight polyethylene or high density polyethylene. In some embodiments, carbon fiber is combined with polyethylene. Additional useful polymers are described below. Ceramics useful for making medical devices of the present invention include, but are not limited to aluminum oxide, calcium phosphates, hydroxyapatite, zirconium oxide, silicon oxide.

In some other embodiments, the implantable portion of the structural support comprises an osteoconductive matrix. The matrix material can be conducive to bone growth. This can be desirable for materials such as teeth and artificial bone graft sections, and the like. Alternatively, when the implantable sections are load bearing and formed, e.g., of stainless steel, these implantable sections can be desirable when formed with a coating of a compound or composition of the present invention. In that event, it is desirable to also provide a separate matrix material conducive to forming new bone growth.

In some embodiments, the matrix comprises particles of porous materials. The pores are preferred to be of a dimension to permit progenitor cell migration into the matrix and subsequent differentiation and proliferation. In some embodiments, the pore size of the matrix is at least 5 μm, e.g., at least 5, 10, 15, 20, 25, 30, 40, 50, 60, 70, 80, 90, 100, 110, 115, 120, 125, 150, 175, 200, 250, 300, 400, 450, 500, 550, 600, 650, 700, 750, 800, 850, 900, 950, 1000, 1250, 1500, 1750 or 2000 μm. In some embodiments, the scaffold or matrix comprises a mesh structure, a foam structure, a sponge structure, or a fiber structure.

A scaffold or matrix for use in a device as described herein can comprise a synthetic and/or biologic material. In some embodiments, the scaffold or matrix comprises a naturally occurring polymer, a synthetic biodegradable polymer, a synthetic nonbiodegradable polymer, a bioceramic, a bioglass, a bioactive glass, a biocomposite, or combinations thereof natural and synthetic polymers, bioceramics, and bioglasses for use in scaffolds are known in the art. See, e.g., Dhandayuthapani et al., International Journal of Polymer Science, volume 2011, article ID 290602 (2011), incorporated by reference herein. Natural polymers include, but are not limited to, proteins (e.g., silk, collagen, gelatin, fibrinogen, elastin, keratin, actin, and myosin), polysaccharides (e.g., cellulose, amylose, dextran, chitin, chitosan, and glycosaminoglycans), and polynucleotides (e.g., DNA and RNA). Synthetic polymers include, but are not limited to, PLA, PGA, PLLA, PLGA, PCL, PLDLA, PDS, PGCL, PEA, PCA, PDLLA, PEU, and PBT. Bioceramics and bioactive glasses include, but are not limited to, HAP, TCP, CP ceramics, BCP, TCP, and 45S5. In some embodiments, the scaffold or matrix is a hydrogel scaffold, a fibrous scaffold, a microsphere scaffold, a polymer-bioceramic composite scaffold, or an acellular scaffold.

In some embodiments, suitable matrixes include those comprising composite biomaterials having a sponge-like structure such as those containing, e.g., phosphophoryn and/or collagen as taught in Takashi Saito's U.S. Publication No. 2006/0188544, published Aug. 24, 2006, the subject matter of which is hereby incorporated by reference. Such coatings include, for example, the single and multilayer coatings taught in U.S. Publication No. 2006/0204542 to Zongtao Zhang et al, published Sep. 14, 2006, as well as those in U.S. Pat. Nos. 6,949,251, 5,298,852, 5,939,039, and 7,189,263 and can be made by conventional methods including the methods taught therein, the subject matter of which is hereby incorporated by reference.

In some embodiments, the matrix is an osteoconductive matrix. In some embodiments, the osteoconductive matrix includes an osteoinductive agent such as bone allograft, bone autograft, bone marrow aspirate, demineralized bone or periodontal ligament cells or combinations thereof. In some other embodiments, the osteoconductive matrix can be a calcium salt, calcium sulfate, biphasic calcium phosphate, calcium phosphate, a calcium phosphate cement, hydroxyapatite, coralline based hydroyxapatite (HA), dicalcium phosphate, tricalcium phosphate (TCP), calcium carbonate, collagen, plaster of Paris, phosphophoryn, a borosilicate, a biocompatible ceramic, a calcium phosphate ceramic, polytetrafluoroethylene, sulfate salt, borosilicate, bioactive glass, Mastergraft variant, Vitoss variant, cement hydrogel, or combinations thereof one of skill in the art will appreciate that other osteconductive matrices and osteoinductive agents are useful in the present invention.

In some embodiments, the medical devices described herein include both a composition described herein and an additional therapeutic agent. Suitable additional therapeutic agents include the combinations discussed, above. For example, the medical devices can include a composition described herein in combination with an anabolic agent. In some embodiments, a medical device described herein include a composition described here in combination with a bone morphogenic protein (BMP) or a BMP agonist. In some embodiments, the BMP is selected from the group consisting of BMP2, BMP7, BMP4, BMP5, and BMP6. In some embodiments, the BMP agonist is a compound described in Vrijens K, et al. PLoS One. 2013; 8(3):e59045, the contents of which is incorporated by reference for all purposes.

### VII. Particular Embodiments of the Present Disclosure

Embodiment 1. A sterilized composition for bone formation, comprising:
a) a scaffold comprising hydroxyapatite and tricalcium phosphate in a ratio of from 0/100 to 15/85, collagen, and bioactive glass, wherein the bioactive glass is uniformly dispersed in both interior and surface portions of the scaffold; and
b) a compound of Formula I:

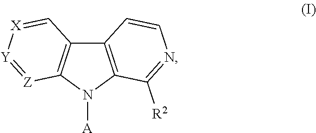

or a salt, hydrate, prodrug, or isomer thereof, wherein
X is selected from CR^{3b} and N, wherein N is optionally oxidized to the corresponding N-oxide;
Y is selected from CR^{3c} and N, wherein N is optionally oxidized to the corresponding N-oxide;
Z is selected from CR^{3d} and N, wherein N is optionally oxidized to the corresponding N-oxide,
provided that at least one of X, Y, and Z is N or the corresponding N-oxide;
A is

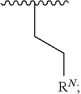

R^{N} is selected from the group consisting of heterocyclyl and heteroaryl, wherein the heterocyclyl moiety is selected from monocyclic, fused bicyclic, and bridged cyclic, the monocyclic heterocyclyl comprising from 4 to 7 ring members, the fused bicyclic and bridged bicyclic heterocyclyl comprising from 7 to 10 ring members, each heterocyclyl moiety having from 1 to 3 heteroatoms as ring members selected from N, O, and S, wherein each heterocyclyl moiety comprises at least one nitrogen atom as a ring member and is optionally substituted with from 1 to 3 R⁶ moieties,
the heteroaryl moiety comprises from 5 to 10 ring members, wherein at least one ring member is a nitrogen atom and is optionally substituted with from 1 to 3 R⁶ moieties, each R², R^{3b}, R^{3c} and R^{3d} is independently selected from the group consisting of H, halogen, C₁₋₆ alkyl, C₁₋₆ haloalkyl, C₂₋₆ alkenyl, C₂₋₆ alkynyl, C₁₋₆ alkoxy, C₁₋₆ haloalkoxy, C₁₋₆ alkyl-OH, —O—C₁₋₆ alkyl-OH, C₃₋₆ cycloalkyl-C₁₋₄ alkoxy, and —OH;
R⁶ is selected from the group consisting of —OH, C₁₋₃ alkyl, C₁₋₃ alkyl-OH, —O—C₁₋₃ alkyl, C₃₋₄ heteroalkyl, C₁₋₃ haloalkyl, —O—C₁₋₃ haloalkyl, halogen, and oxo;
and wherein the composition is sterilized and packaged.

Embodiment 2. A composition for bone formation, comprising a scaffold of hydroxyapatite (HA) and tricalcium phosphate (TCP) in a ratio of from 0/100 to 15/85, collagen, and bioactive glass, wherein the bioactive glass is uniformly dispersed in both interior and surface portions of the scaffold, and wherein the composition is sterilized and packaged.

Embodiment 3. The composition of embodiment 1 or 2, wherein the scaffold is in the form of granules, a strip, block, sphere, putty, or liquid cement.

Embodiment 4. The composition of embodiment 1 or 2, wherein the hydroxyapatite and tricalcium phosphate are in a ratio of about 0/100, 5/95 or 10/90.

Embodiment 5. The composition of embodiment 1 or 2, wherein the bioactive glass has a particle size of from about 50 microns to about 500 microns.

Embodiment 6. The composition of embodiment 1 or 2, wherein the bioactive glass has a particle size of from about 90 microns to about 250 microns.

Embodiment 7. The composition of embodiment 1 or 2, wherein the composition is moldable.

Embodiment 8. The composition of embodiment 1 or 2, wherein the composition has been sterilized using a method selected from the group consisting of gamma irradiation, E-beam irradiation, UV irradiation, steam, dry heat, plasma, and chemical sterilization (ether, ethanol, iodine, or PAA).

Embodiment 9. The composition of embodiment 1, wherein said compound has a formula Ia, Ib, Ic, or Id:

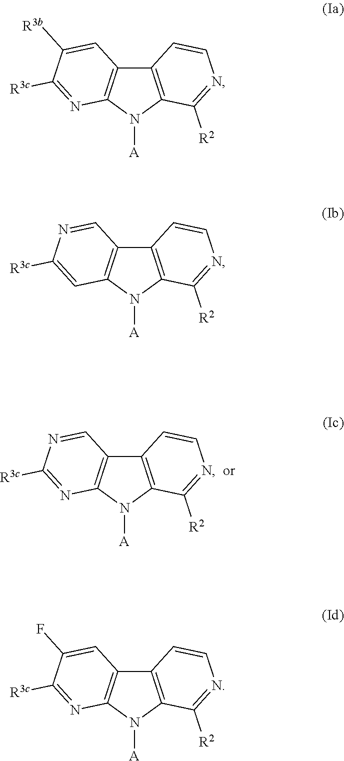

Embodiment 10. The composition of embodiment 9, wherein R² is selected from the group consisting of H, halogen, C₁₋₆ alkyl, C₁₋₆ haloalkyl, C₁₋₆ alkoxy, and C₁₋₆ haloalkoxy.

Embodiment 11. The composition of embodiment 9, wherein R² is selected from the group consisting of halogen, C₁₋₆ alkyl, C₁₋₆ haloalkyl, and C₁₋₆ alkoxy.

Embodiment 12. The composition of embodiment 9, wherein R² is C₁₋₆ alkyl or C₁₋₆ haloalkyl.

Embodiment 13. The composition of embodiment 9, wherein R² is CH₃ or CF₃.

Embodiment 14. The composition of embodiment 9, wherein R² is CH₃.

Embodiment 15. The composition of embodiment 9, wherein R² is CF₃.

Embodiment 16. The composition of embodiment 1, wherein R^{N} is heterocyclyl or heteroaryl.

Embodiment 17. The composition of embodiment 16, wherein R^{N} is a monocyclic heterocyclyl.

Embodiment 18. The composition of embodiment 17, wherein R^{N} is

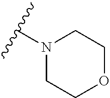 [SMILES: *N1CCOCC1]

Embodiment 19. The composition of embodiment 18, wherein said compound is selected from the group consisting of:

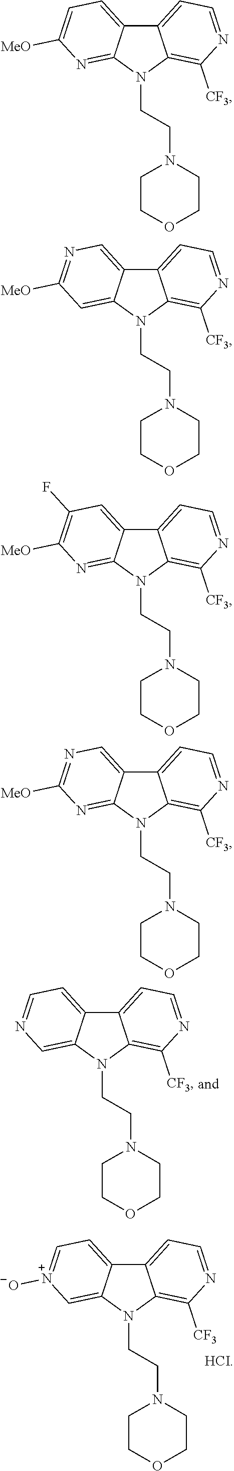

or salts, hydrates, or prodrugs thereof.

Embodiment 20. The composition of any one of embodiments 1 to 19, further comprising an additional bone-forming agent.

Embodiment 21. The composition of embodiment 20, wherein the bone-forming agent is selected from the group consisting of P15, BMP2, BMP7, BMP4, PTH, anti-sclerostin antibodies, anti-sclerostins and antiresorptives.

Embodiment 22. A method of promoting bone formation a subject in need thereof, comprising locally administering to the subject an effective amount of a composition of any one of embodiments 1 to 21, thereby promoting bone formation in the subject.

Embodiment 23. The method of embodiment 22, wherein the bone formation is promoted at a surgical site of injury or localized condition.

Embodiment 24. The method of embodiment 23, wherein the bone formation is promoted at a surgical site selected from the group consisting of a bone fracture and weakened bone.

Embodiment 25. The method of embodiment 23, wherein the subject is in need of a spinal fusion, arthrodesis or an orthopedic or periodontal synthetic bone graft or implant.

Embodiment 26. The method of embodiment 22, wherein the subject has a low bone mass/density condition, a bone fracture, or periodontal disease.

Embodiment 27. The method of embodiment 26, wherein the low bone mass condition is selected from osteoporosis, osteopenia, osteogenesis imperfecta (OI), osteoporosispseudoglioma syndrome (OPPG), and secondary low bone condition.

Embodiment 28. The method of embodiment 27, wherein the low bone mass condition is selected from the group consisting of osteoporosis, osteopenia, and osteoporosispseudoglioma syndrome (OPPG).

Embodiment 29. The method of embodiment 22, further comprising administering to the subject additional osteoconductive matrix components.

Embodiment 30. The method of embodiment 29, wherein the additional osteoconductive matrix components are selected from the group consisting of bone allograft, bone autograft, and periodontal ligament cells.

Embodiment 31. The method of embodiment 30, wherein the additional osteoconductive matrix components are selected from the group consisting of a calcium salt, calcium sulfate, calcium phosphate, a calcium phosphate cement, hydroxyapatite, coralline based hydroyxapatite (HA), dicalcium phosphate, tricalcium phosphate (TCP), calcium carbonate, collagen, plaster of Paris, phosphophoryn, a borosilicate, a biocompatible ceramic, a calcium phosphate ceramic, demineralized bone matrix, biphasic calcium phosphate, biocomposite, tantalum, titanium, polytetrafluoroethylene, sulfate salt, hydrogel, and combinations thereof.

Embodiment 32. The method of embodiment 22, wherein the composition is administered sequentially or in combination with an antiresorptive drug.

Embodiment 33. The method of embodiment 32, wherein the composition is locally administered to a subject treated with the antiresorptive drug or has previously been treated with the antiresorptive drug.

Embodiment 34. The method of embodiment 32, wherein the antiresorptive drug is selected from the group consisting of denosumab, prolia, a RankL inhibitor, a bisphosphonate, a selective estrogen receptor modulator (SERM), calcitonin, a calcitonin analog, Vitamin D, a Vitamin D analog, and a cathepsin K inhibitor.

Embodiment 35. A method of treating bone loss in a subject in need thereof, comprising administering to the subject a therapeutically effective of a composition of embodiment 36 in series or in combination with an antiresorptive agent, thereby treating bone loss in a subject.

### VIII. Examples

The following examples are offered to illustrate, but not to limit the claimed invention.

Compounds used in the Examples below, and useful in the compositions and methods described herein can be obtained using methods described in WO2020/037001, WO2020/037004, and WO2014/153203, the contents of which are incorporated herein by reference.

### Example 1: Preparation of Scaffold

This example provides instructions for preparing the scaffold described in the present disclosure.

Step 1—Addition of bioactive glass. For each cc of scaffold (comprising HA, TCP and collagen, available from commercial sources), hydrate with up to 1.48 mL dd-water/cc scaffold such that scaffold is pliable. Add 125 mg bioactive glass (BA) and massage into the pliable scaffold until uniformly distributed throughout. Shape/mold scaffold to desired shape and dimensions (i.e. block, cylinder, strip, sphere, etc) and allow to dry and re-solidify over >24 hours depending on overall size and thickness. Scaffold and BA are now presented as a singular product.

Step 2—Addition of API. Take solid API into water with agitation (i.e. sonication) to give a 1.8 mM solution. For each cc of scaffold+BA, add 666.3 uL 1.8 mM API. Allow API to crystallize and dry within scaffold over >24 hours depending on overall size and thickness.

### Example 2: Bone Formation Assays

Mineralization (crystalline calcium phosphate formation) represents an in vitro model of bone formation. Using an assay in which the amount of mineralization is quantified by measuring total calcium after solubilization of deposited crystalline calcium phosphate, sclerostin was previously shown to inhibit mineralization in MC3T3-E1 (mouse calvarial) osteoblast cells. Li et al., J Bone Miner Res 24:578-588 (2008). Following the protocol described in Li et al., Compositions prepared in accordance with Example 1 or mixtures of the described composition components were assayed for their ability to rescue the inhibition of mineralization by sclerostin in MC3T3 osteoblast cells. This assay was used in the examples listed below.

### Example 3: Scaffold Size & Ratio of Hydroxyapatite (HA) and Tricalcium Phosphate (TCP)

This example illustrates the testing of HA to TCP ratios and the effect of scaffold size on mineralization.

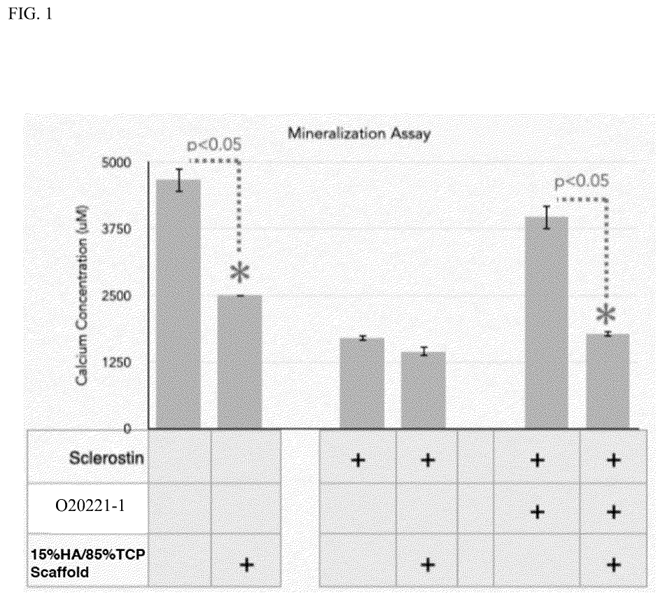

Using the mineralization assay described in Example 2, a scaffold of 15:85 HA to TCP was prepared and tested. FIG. 1 shows ARS mineralization is reduced in presence of 15% HA/85% TCP Scaffold. The first column represents wildtype mineralization, whereby the cells have no additives (sclerostin, O20221-1, scaffold). Addition of ectopic sclerostin inhibits normal mineralization (column 3). The positive control O20221-1 in presence of sclerostin restores normal mineral (compare column 3 and 5). 15% HA/85% TCP scaffold alone is unable produce 100% wildtype mineral (compare column 1 with column 2) and is unable to restore 100% wildtype mineral in presence of sclerostin (column 4). O20221-1 loaded 15% HA/85% TCP scaffold is unable to restore normal mineral (column 6 compared to column 1).

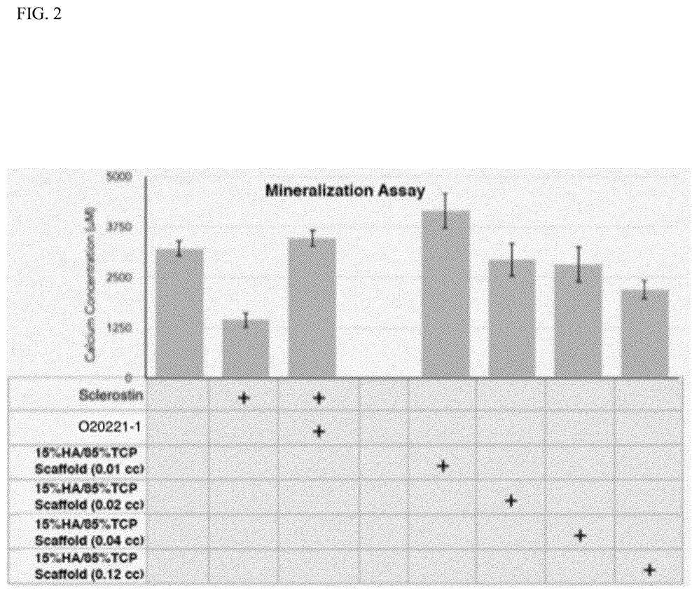

A further test was performed testing the effect the size of the scaffold has on mineralization. FIG. 2 shows ARS mineralization is reduced by increasing 15% HA/85% TCP scaffold size. The first column represents wildtype mineralization, whereby the cells have no additives (sclerostin, O20221-1, scaffold or granules). In column 2, Sclerostin inhibits wildtype mineralization. The addition of O20221-1 in presence of sclerostin restores normal mineral (Column 3). Decreasing the size of the scaffold from 0.12 cc-0.01 cc restores the reduced mineral (column 7) back to wildtype mineral levels (dose response & columns 4-7). 15% HA/85% TCP scaffold shows decrease in mineral as scaffold size increases from 0.01 cc to 0.12 cc (compare column 4 to 1).

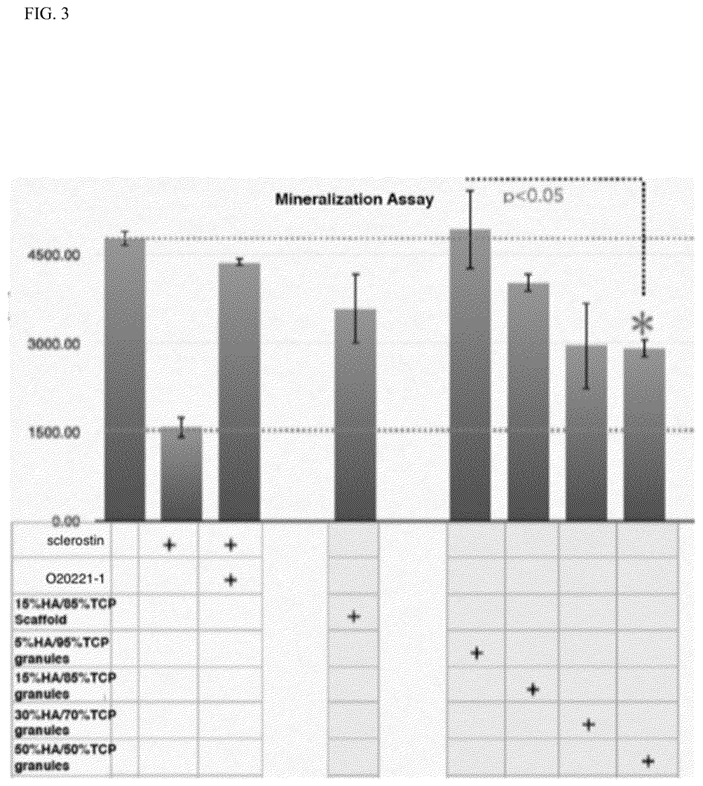

Using the mineralization assay described in Example 2, scaffolds with varying ratios of HA:TCP were tested. FIG. 3 shows ARS mineralization is reduced by increasing percentage of HA content. The first column represents wildtype mineralization, whereby the cells have no additives (sclerostin, O20221-1, scaffold or granules). In column 2, Sclerostin inhibits wildtype mineralization. The addition of O20221-1 in presence of sclerostin restores normal mineral (Column 3). Addition of 15% HA/85% TCP scaffold alone shows a decrease in wild type mineral (compare column 4 to 1). Increasing concentration (percentages from 5-50%) of HA decreases wild type mineral produced (dose response & columns 5-8).

### Example 4: Surprising Increase in Mineralization Provided by Bioactive Glass

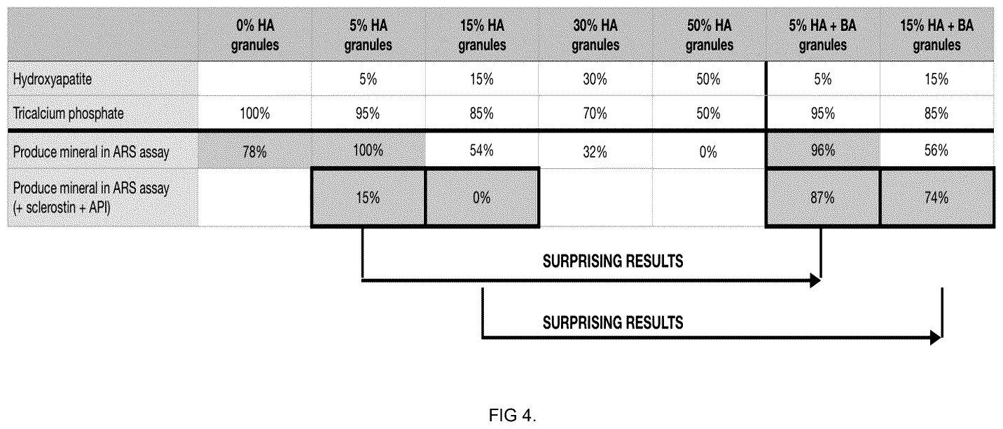

This example describes the results obtained when bioactive glass was included in the compositions/scaffolds described herein. Again, testing was done using the mineralization assay described in Example 2. The Table below summarizes the surprising improvement in mineralization observed when bioactive glass (BA) was included. Results are summarized in FIG. 4. The first 5 columns show the effect of variable HA content from 0-50% on mineralization 100% to 0%. The low mineralization of the scaffold in presence of sclerostin and O20221-1 can be restored by the addition of non-encased bioactive glass (BA) (column 6 & 7 compared to column 2 & 3).

Although the foregoing invention has been described in some detail by way of illustration and example for purposes of clarity of understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A sterilized composition for bone formation, comprising
a) a scaffold comprising hydroxyapatite and tricalcium phosphate in a ratio of from 0/100 to 15/85, collagen, and bioactive glass, wherein the bioactive glass is uniformly dispersed in both interior and surface portions of the scaffold; and
b) a compound of Formula I: 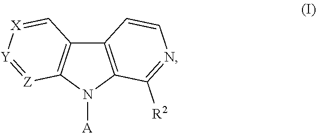
or a salt, hydrate, prodrug, or isomer thereof, wherein
X is selected from CR^{3b} and N, wherein N is optionally oxidized to the corresponding N-oxide;
Y is selected from CR^{3c} and N, wherein N is optionally oxidized to the corresponding N-oxide;
Z is selected from CR^{3d} and N, wherein N is optionally oxidized to the corresponding N-oxide,
provided that at least one of X, Y, and Z is N or the corresponding N-oxide;
A is 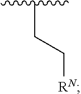
R^{N} is selected from the group consisting of heterocyclyl and heteroaryl, wherein
the heterocyclyl moiety is selected from monocyclic, fused bicyclic, and bridged cyclic, the monocyclic heterocyclyl comprising from 4 to 7 ring members, the fused bicyclic and bridged bicyclic heterocyclyl comprising from 7 to 10 ring members, each heterocyclyl moiety having from 1 to 3 heteroatoms as ring members selected from N, O, and S, wherein each heterocyclyl moiety comprises at least one nitrogen atom as a ring member and is optionally substituted with from 1 to 3 R⁶ moieties,
the heteroaryl moiety comprises from 5 to 10 ring members, wherein at least one ring member is a nitrogen atom and is optionally substituted with from 1 to 3 R⁶ moieties,
each R², R^{3b}, R^{3c} and R^{3d} is independently selected from the group consisting of H, halogen, C₁₋₆ alkyl, C₁₋₆ haloalkyl, C₂₋₆ alkenyl, C₂₋₆ alkynyl, C₁₋₆ alkoxy, C₁₋₆ haloalkoxy, C₁₋₆ alkylOH, O—C₁₋₆ alkylOH, C₃₋₆ cycloalkyl-C₁₋₄alkoxy, and —OH;
R⁶ is selected from the group consisting of —OH, C₁₋₃ alkyl, C₁₋₃ alkylOH,
—O—C₁₋₃ alkyl, C₃₋₄ heteroalkyl, C₁₋₃ haloalkyl, —O—C₁₋₃ haloalkyl, halogen, and oxo;
and wherein the composition is sterilized and packaged.

2. A composition for bone formation, comprising a scaffold of hydroxyapatite (HA) and tricalcium phosphate (TCP) in a ratio of from 0/100 to 15/85, collagen, and bioactive glass, wherein the bioactive glass is uniformly dispersed in both interior and surface portions of the scaffold, and wherein the composition is sterilized and packaged.

3. The composition of claim 1, wherein the scaffold is in the form of granules, a strip, block, sphere, putty, or liquid cement.

4. The composition of claim 1, wherein the hydroxyapatite and tricalcium phosphate are in a ratio of about 0/100, 5/95 or 10/90.

5. The composition of claim 1, wherein the bioactive glass has a particle size of from about 50 microns to about 500 microns.

6. The composition of claim 1, wherein the bioactive glass has a particle size of from about 90 microns to about 250 microns.

7. (canceled)

8. The composition of claim 1, wherein the composition has been sterilized using a method selected from the group consisting of gamma irradiation, E-beam irradiation, UV irradiation, steam, dry heat, plasma, and chemical sterilization (ether, ethanol, iodine, or PAA).

9. The composition of claim 1, wherein said compound has a formula Ia, Ib, Ic, or Id:

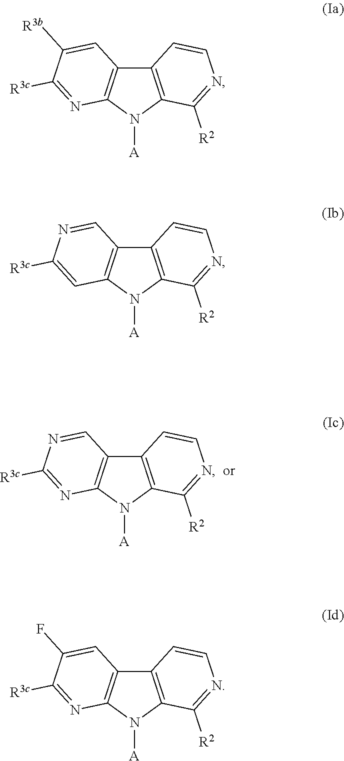

10. The composition of claim 9, wherein R² is selected from the group consisting of H, halogen, C₁₋₆ alkyl, C₁₋₆ haloalkyl, C₁₋₆ alkoxy, and C₁₋₆ haloalkoxy.

11. The composition of claim 9, wherein R² is selected from the group consisting of halogen, C₁₋₆ alkyl, C₁₋₆ haloalkyl, and C₁₋₆ alkoxy.

12. The composition of claim 9, wherein R² is C₁₋₆alkyl or C₁₋₆ haloalkyl.

13. The composition of claim 9, wherein R² is CH₃ or CF₃.

14. (canceled)

15. The composition of claim 9, wherein R² is CF₃.

16. The composition of claim 1, wherein R^{N}is heterocyclyl or heteroaryl.

17. The composition of claim 16, wherein R^{N} is a monocyclic heterocyclyl.

18. The composition of claim 17, wherein R^{N} is

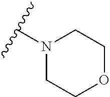 [SMILES: *N1CCOCC1]

19. The composition of claim 17, wherein said compound is selected from the group consisting of: 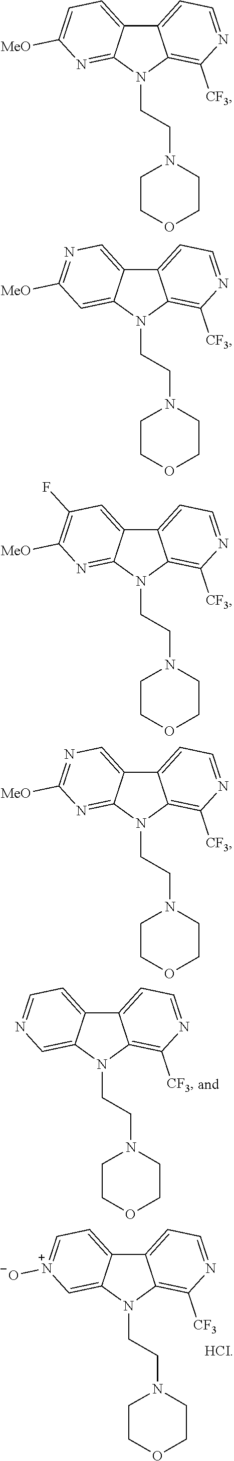
or salts, hydrates, or prodrugs thereof.

20. The composition of claim 1, further comprising an additional bone-forming agent.

21. (canceled)

22. A method of promoting bone formation a subject in need thereof, comprising locally administering to the subject an effective amount of a composition of claim 1, thereby promoting bone formation in the subject.

23.-34. (canceled)

35. A method of treating bone loss in a subject in need thereof, comprising administering to the subject a therapeutically effective of a composition of claim 1 in series or in combination with an antiresorptive agent, thereby treating bone loss in a subject.
